(19) United States
(12) Patent Application Publication
(10) Pub. No.: US 2024/0165187 A1
(43) Pub. Date: May 23, 2024

(54) ANTIVIRAL OLIVE EXTRACT COMPOSITIONS AND METHODS

(71) Applicants: Roberto Crea, Hayward, CA (US); Yohei Takeda, Obihiro (JP); Haruko Ogawa, Obihiro (JP)

(72) Inventors: Roberto Crea, Hayward, CA (US); Yohei Takeda, Obihiro (JP); Haruko Ogawa, Obihiro (JP)

(21) Appl. No.: 18/207,469

(22) PCT Filed: Dec. 7, 2021
(86) PCT No.: PCT/US2021/062213

§ 371 Date: Jun. 8, 2023

Related U.S. Application Data
(63) Continuation-in-part of application No. 17/326,146, filed on May 20, 2021, now Pat. No. 11,951,143.
(60) Provisional application No. 63/122,893, filed on Dec. 8, 2020.

(51) Int. Cl. A61K 36/63; A61K 31/05; A61P 31/16

(52) U.S. Cl. CPC A61K 36/63; A61K 31/05; A61P 31/16; A61K 2236/15; A61K 2236/331; A61K 2236/51

(57) ABSTRACT

An olive component composition with anti-viral activity is disclosed. The olive component composition can be used to prevent, treat, or ameliorate symptoms of, viral infection.

This application claims priority to U.S. Provisional Patent Application Ser. No. 63/122,893, filed on Dec. 8, 2020, and U.S. patent application Ser. No. 17/326,146, filed on May 20, 2021, the contents of which are expressly incorporated herein by reference.

### FIELD

This application generally relates to anti-viral compositions and, in particular, to olive extracts with anti-viral effect and method of use thereof.

### BACKGROUND

Viral infections, such as the severe acute respiratory syndrome coronavirus 2 (SARS-CoV-2) pandemic, remain a serious problem. In addition to the existing measures like vaccination, medication, and inactivation of environmental viruses using chemical disinfectants, there is an increasing demand for the development of auxiliary approaches for the preventions of viral infection and aggravation of symptoms.

The present application addresses this demand by providing natural-derived compositions as supplemental agents for protection from pathogenic viruses, such as SARS-CoV-2.

### SUMMARY

One aspect of the present application relates to a method for preventing, treating, or ameliorating symptoms, of a viral infection in a subject. The method comprises the step of administering to the subject an effective amount of an olive component composition, wherein the olive component composition comprises one or more polyphenyl compounds selected from the group consisting of hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof.

Another aspect of the present application relates to a method for preventing or reducing surface transmission of a virus. The method comprises the step of applying an effective amount of an olive component composition to a surface for a period of time to inactivate the virus on the surface, wherein the olive component composition comprises one or more polyphenyl compounds selected from the group consisting of hydroxytyrosol, tyrosol, tyrosol esters of elenolic acid, such as oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof.

Another aspect of the present application relates to an antiviral composition. The composition comprises an olive component composition, wherein the olive component composition comprises: (1) one or more polyphenyl compounds selected from the group consisting of hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof, (2) propanoic acid, and optionally (3) no syringic acid or syringic acid in an amount of less than 0.01 mg/ml.

Another aspect of the present application relates to an antiviral composition formulated for inhalation. The antiviral composition comprises an olive component composition, wherein the olive component composition comprises: one or more polyphenyl compounds selected from the group consisting of hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof, wherein the antiviral composition is formulated in an aerosol for inhalation.

### BRIEF DESCRIPTION OF THE DRAWINGS

FIG. 1 is a schematic illustration of an exemplary process for preparing a hydroxytyrosol (HT)-rich aqueous olive pulp extract (HIDROX®).

FIG. 2, panel A is a schematic illustration of an experimental protocol for evaluating the time- and concentration-dependent virucidal activity of HIDROX® against severe acute respiratory syndrome coronavirus 2 (SARS-CoV-2).

FIG. 3 shows the time- and concentration-dependent virucidal activity of HIDROX® against SARS-CoV-2. In the experiments depicted in FIG. 3, SARS-CoV-2 was mixed with HIDROX®. The final concentrations of HIDROX® in the mixture were 0.45, 0.90, and 4.50 mg/mL (FIG. 3, panel A) or 5.63 and 11.25 mg/mL (FIG. 3, panel B). As a diluent control, PBS was mixed with the viral suspension. The mixtures were incubated at 25° C. for 0.5-24 h (Panel A) or 5 min (Panel B) after which the viral titers were evaluated. The detection limits for the viral titers are 1.25 log₁₀ at a 50% tissue culture infective dose (TCID₅₀)/mL in the PBS and HIDROX® (0.45 mg/mL) groups, 2.25 log₁₀ TCID₅₀/mL in the HIDROX® (0.9, 4.50, and 5.63 mg/mL) groups, and 3.25 log₁₀ TCID₅₀/mL in the HIDROX® (11.25 mg/mL) group. Results are indicated as mean±SD (n=3-4 per group). Student's t-test was performed to evaluate the statistically significant difference between the PBS and HIDROX® groups; * p<0.05; ** p<0.01; *** p<0.001.

FIG. 4 shows an experimental protocol for evaluating the time-dependent virucidal activity of HIDROX® against four SARS-CoV-2 variant strains, namely the UK variant, the Brazilian variant the South African variant and the Delta variant.

FIG. 5 shows the time-dependent virucidal activity of HIDROX® against the four SARS-CoV variants according to the experimental protocol depicted in FIG. 4. Student's t-test was performed to evaluate the statistically significant difference between the PBS group and each test group; ** p<0.01; *** p<0.001.

FIG. 6, panel A depicts an experimental protocol for evaluating the comparative virucidal activities of HIDROX® and HT against SARS-CoV-2. In FIG. 6, HIDROX® and HT were mixed with SARS-CoV-2 at 0.90 mg/ml, inoculated to cells and incubated at 25° C. for 3 or 24 hr. The final concentration of HIDROX® in the mixture was 0.90 mg/mL; the final concentration of HT in the mixtures were 0.90 mg/mL and 0.05. PBS was mixed with the viral suspension as the diluent control. Following the incubation period, viral titers were determined. FIG. 6, panel B shows the results of the experiment depicted in FIG. 6, panel A. In panel B, the detection limits of viral titer are 1.25 log₁₀ 50% tissue culture infective dose (TCID₅₀)/mL in the PBS and HT (0.05 mg/mL) groups and 2.25 log₁₀ TCID₅₀/mL in the HIDROX® (0.9 mg/mL) and HT (0.90 mg/mL) groups. Results are indicated as mean±SD (n=4 per group). Student's t-test was performed to evaluate the statistically significant difference between the PBS group and each test group; * p<0.05; ***p<0.001.

FIG. 7, panel A shows an experimental protocol for examining the direct virucidal activity of HIDROX® against SARS-CoV-2 at various concentrations (6.25-100 mg/ml). FIG. 7, panel B shows the virucidal activity of HIDROX® at various concentrations in accordance with the schematic diagram in FIG. 7.

FIG. 8 shows an experimental protocol for evaluating the inhibitory effect of HIDROX® (pH 7.4) on virus proliferation in host cells.

FIG. 9 shows the results of the experiment described in FIG. 8.

FIG. 10 shows an experimental protocol for examining the direct virucidal activity of HIDROX® against SARS-CoV-2 at low concentrations (0.5-5.0 mg/ml).

FIG. 11 shows the results of the experiment described in FIG. 10.

FIG. 12 shows the cytotoxicity of HT (0-50 μg/ml) compared to HIDROX® (0-250 μg/ml).

FIG. 13, panel A shows an experimental protocol for comparing the virucidal activities of HIDROX® and HT against SARS-CoV-2 at 1.0 mg/ml and 0.05 mg/ml. FIG. 13, panel B shows the result of the experiment described in panel A.

FIG. 14 shows the time- and concentration-dependent virucidal activity of HIDROX®-containing cream against SARS-CoV-2. SARS-CoV-2 was covered by 0%, 2%, 5%, and 10% HIDROX®-containing cream-coated films and incubated for 10 min-6 h at 25° C. After the predetermined reaction times, the viral solutions were recovered and the viral titers were determined. The detection limit of the viral titer is 1.25 log₁₀ 50% tissue culture infective dose (TCID₅₀)/mL in all groups. Results are indicated as mean±SD (n=4-8 per group). Student's t-test was performed to evaluate the statistically significant differences between the 0% HIDROX® group and other groups; * p<0.05; ** p<0.01; *** p<0.001.

FIG. 15 shows the impact of HIDROX® and HT on SARS-CoV-2 proteins. As shown in FIG. 15, panel A, SARS-CoV-2 was mixed with HIDROX® or HT. The final concentrations of HIDROX® and HT in each mixture was 0.90 mg/mL. PBS was mixed with the SARS-CoV-2 as a diluent control. The mixtures were incubated at 25° C. for 0 (no reaction time) or 24 h, and then Western blotting was performed. In FIG. 15, panel A, the images on the left, middle, and right show the results of Western blotting to detect S protein S1 subunit, S protein S2 subunit, and N protein, respectively. In FIG. 15, panel B, the recombinant S protein RBD or N protein was mixed with PBS, HIDROX®, or HT. The final concentration of HIDROX® and HT in each mixture was 0.90 mg/mL. The final concentration of recombinant viral proteins in each mixture was approximately 10 μg/mL. The mixtures were incubated at 25° C. for 0 (no reaction time) or 24 h and then Western blotting was performed. The images on the left and right show the results of Western blotting to detect S protein RBD and N protein, respectively.

FIG. 16 shows the impact of HIDROX® or HT on carbohydrate chains expressed on the S protein. In this experiment, glycosylated and deglycosylated forms of recombinant S protein S1 subunit, RBD, and S2 subunit were mixed with PBS, HIDROX®, or HT. The final concentration of HIDROX® and HT in each mixture was 0.90 mg/mL. The final concentration of recombinant S proteins in the mixtures was approximately 10 μg/mL. The mixtures were incubated at 25° C. for 24 h and then Western blotting was performed. The images on the left, middle, and right show the glycosylated and unglycosylated forms of the S1 subunit, RBD, and S2 subunit, respectively.

FIG. 17 shows the impact of HIDROX® and HT on SARS-CoV-2 genome. In this experiment, SARS-CoV-2 was mixed with HIDROX® or HT at a concentration of 0.90 mg/mL. As a diluent control, PBS was mixed with SARS-CoV-2. The mixtures were incubated at 25° C. for 0 (no reaction time) or 24 h. Then real-time RT-PCR was performed and Ct values were determined. The results are indicated as mean±SD (n=4 per group). Student's t-test was performed to evaluate the statistically significant differences between the PBS group and each test solution group; ** p<0.01; *** p<0.001.

FIG. 18 shows the cytotoxicity of HT (0-500 μg/ml) compared to HIDROX® (0-500 μg/ml) in MDCK cells, the host cells for influenza (H1N1) in the experiment described in FIG. 19.

FIG. 19, panel A shows an experimental protocol for evaluating the virucidal activity of HIDROX® against influenza-infected MDCK cells at various different concentrations and incubation periods. FIG. 19, panel B shows the result of the experiment described in panel A.

FIG. 20 shows GC chromatographic profiles of (A) OLIVENOL™ (in column 9.0 μg), vegetation waters (B) CR43 (10.0 μg) and (C) CR21 (8.0 μg), and (d) polar fraction from 100 ml of Moraiolo extra-virgin olive oil (17.8 μg). Peaks: 1, tyrosol; 2, vanillic acid; 3, hydroxytyrosol (i.e. HT); 4,3,4-dihydroxybenzoic acid; 5, citric acid; 6, syringic acid; 7, gallic acid.

FIG. 21 shows GC chromatographic profiles of (A) not treated OVW at time O (in column 12.0 μg), (B) not treated OVW after 6 months (12.0 μg) and (C) treated OVW after 6 months (10.0 μg). Peaks: 1, tyrosol; 2, vanillic acid; 3, hydroxytyrosol; 4,3,4-dihydroxybenzoic acid; 5, citric acid; 6, syringic acid; 7, gallic acid; 8, caffeic acid; 9,3-hydroxy, 4-methoxy-phenylacetic acid; 10, gentisic acid.

FIG. 22, panel A shows an experimental protocol for evaluating inhibitory effects of Olivenol Plus® Essence Liquid on suppression of SARS-CoV-2 proliferation in cells. FIG. 22, panel B shows the results of the experiment in FIG. 22, panel A.

FIG. 23, panel A shows an experimental protocol for evaluating inhibitory effects of HIDROX® on suppression of SARS-CoV-2 proliferation in cells. FIG. 23, panel B shows the results of the experiment in FIG. 23, panel A.

FIG. 24 shows an experimental protocol for evaluating antiviral activity of cells pre-treated with Olivenol Plus® Essence Liquid on suppression of SARS-CoV-2 proliferation in cells. FIG. 24, panel B shows the results of the experiment in FIG. 24, panel A.

FIG. 25 shows an experimental protocol for evaluating antiviral activity of cells pre-treated with HIDROX® on suppression of SARS-CoV-2 proliferation in cells. FIG. 25, panel B shows the results of the experiment in FIG. 25, panel A.

FIG. 26, panel A shows an experimental protocol for evaluating direct virucidal activity of Olivenol Plus® Essence Liquid on suppression of SARS-CoV-2 proliferation in cells. FIG. 26, panel B shows the results of the experiment in FIG. 26, panel A.

FIG. 27, panel A shows an experimental protocol for evaluating direct virucidal activity of HIDROX® on suppression of SARS-CoV-2 proliferation in cells. FIG. 27, panel B shows the results of the experiment in FIG. 27, panel A.

FIG. 28, panel A shows an experimental protocol for evaluating the effects of acidic pH on HIDROX®'s virucidal activity in suppressing SARS-CoV-2 proliferation in cells. FIG. 28, panel B shows the results of the experiment in FIG. 28, panel A.

FIG. 29, panel A shows an experimental protocol for evaluating the effects of HIDROX® in suppressing the proliferation of the Delta variant of SARS-CoV-2 in cells. FIG. 29, panel B shows the results of the experiment in FIG. 29, panel A.

### DETAILED DESCRIPTION

The disclosure and accompanying drawings will now be discussed to enable one skilled in the art to practice the invention described herein. The skilled artisan will understand, however, that the embodiments described below can be practiced without employing every specific detail, or that they can be used for purposes other than those described herein. Indeed, they can be modified and can be used in conjunction with products and techniques known to those of skill in the art considering the present disclosure. The drawings and descriptions are intended to be exemplary of various aspects of the disclosure and are not intended to narrow the scope of the appended claims. Furthermore, it will be appreciated that the drawings may show aspects of the disclosure in isolation and the elements in one figure may be used in conjunction with elements shown in other figures.

It will be appreciated that reference throughout this specification to aspects, features, advantages, or similar language does not imply that all the aspects and advantages may be realized with the present disclosure or realized are in any single embodiment of the disclosure. Rather, language referring to the aspects and advantages should be understood to mean that a specific aspect, feature, advantage, or characteristic described in connection with an embodiment is included in at least one embodiment of the present disclosure. Thus, discussion of the aspects and advantages, and similar language, throughout this specification may, but do not necessarily, refer to the same embodiment.

The described aspects, features, advantages, and characteristics of the disclosure may be combined in any suitable manner in one or more further embodiments. Furthermore, one skilled in the relevant art will recognize that the embodiments may be practiced without one or more of the specific aspects or advantages of a particular embodiment. In other instances, additional aspects, features, and advantages may be recognized and claimed in certain embodiments that may not be present in all embodiments of the disclosure.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

### I. Definitions

Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present disclosure. The described aspects and embodiments of the application are not limited to the methods and materials described.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The terms “a”, “an,” or “the” as used in the specification and claims, unless clearly indicated to the contrary, should be understood to mean “at least one” or “one or more,” unless the content clearly dictates otherwise.

The phrase “and/or,” as used herein in the specification and claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. Further, if the disclosure describes “a composition comprising A and B”, the disclosure also contemplates the alternative embodiments “a composition consisting of A and B” and “a composition consisting essentially of A and B”.

Where ranges are given, endpoints are included. Further, it is to be understood that unless otherwise indicated or otherwise evident from the context and understanding of one of ordinary skill in the art, values that are expressed as ranges can assume any specific subrange within the stated ranges in different embodiments of the invention, or any subrange defined by any pair of integers within a stated range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

As used herein, the phrases “administered in combination” or “combined administration” means that two or more agents are administered to a subject at the same time or within an interval such that there may be an overlap of an effect of each agent on the patient. In some embodiments, they are administered within about two weeks, one week, 2-5 days, one week, 60 min, 30 min, 15 min, 10 min, 5 min, or 1 min of one another. In some embodiments, the administrations of the agents are spaced sufficiently closely together such that a combinatorial (e.g., a synergistic) effect is achieved.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “control individual” is an individual who is not afflicted with the same virus as the individual being treated, who is about the same age as the individual being treated (to ensure that the stages of the disease in the treated individual and the control individual(s) are comparable). The individual (also referred to as “patient” or “subject”) being treated may be a fetus, infant, child, adolescent, or adult human.

The phrases “effective amount”, “therapeutically effective amount” and “pharmacologically effective amount” are used interchangeably with reference to an amount of an agent that is sufficient to effect beneficial or desired results, for example, clinical results, and, as such, an “effective amount” depends upon the context in which it is being applied. For example, in the context of administering an antiviral composition for a SARS-CoV-2 infection, an effective amount refers to an amount sufficient to provide antiviral activity against SARS-CoV-2, as compared to responses (or lack thereof) obtained without administration of the antiviral composition.

The terms, “improve”, “increase,” “reduce”, “ameliorate,” as used in this context, indicate values or parameters relative to a baseline measurement, such as a measurement in the same individual prior to initiation of the treatment described herein, or a measurement in a control individual (or multiple control individuals) in the absence of the treatment.

The phrase “pharmaceutically acceptable” refers to those compounds, materials, compositions, and/or dosage forms which are, within the scope of sound medical judgment, suitable for use in human beings and animals without excessive toxicity, irritation, allergic response, or other problem or complication, commensurate with a reasonable benefit/risk ratio when administered to an animal or a human, as appropriate.

The term “pharmaceutically acceptable carrier” refers to pharmaceutically-acceptable materials, compositions or vehicles, including any and all solvents, solubilizers, fillers, diluents, stabilizers, surfactants, binders, absorbents, bases, buffering agents, excipients, lubricants, controlled release vehicles, diluents, emulsifying agents, encapsulating materials, humectants, lubricants, gels, dispersion media, coatings, antibacterial or antifungal agents, isotonic and absorption delaying agents, and the like, compatible with pharmaceutical administration. The use of such carriers and agents for pharmaceutically active substances is well-known in the art.

As used herein, the term “preventing” refers to partially or completely delaying onset of a viral infection, such as a SARS-CoV-2 infection, disease, disorder and/or condition thereof, partially or completely delaying onset of one or more symptoms, features, or clinical manifestations of a viral infection, disease, disorder, and/or condition thereof; partially or completely delaying onset of one or more symptoms, features, or manifestations of a viral infection, disorder, and/or condition thereof, partially or completely delaying progression from viral infection, disease, disorder and/or condition thereof, and/or decreasing the risk of developing pathology associated with viral infection, disease, disorder, and/or condition thereof.

As used herein, “SARS-CoV-2” refers to betacoronavirus believed to be of lineage B (sarbecovirus). SARS-CoV-2 was first identified in Wuhan, Hubei province, China, in late 2019. As used herein, the term “SARS-CoV-2” refers to both the “SARS-CoV-2 original strain” and variants of the SARS-CoV-2 original strain.

As used herein, the phrase a “variant of SARS-CoV-2” and “SARS-CoV-2 variant” are used interchangeably with reference to SARS-CoV-2 strains that differ from original or progenitor SARS-CoV-2 strains by one or more nucleotides in the viral genome. Variants of the SARS-CoV-2 original strain may be further divided into subgroups of variants, such as Alpha variants, Beta variants, Delta variants, Delta plus variants, omicron variants and the like, which have accumulated enough mutations to represent separate branches on the family tree.

As used herein, the term “SARS-CoV-2 spike protein,” refers to the spike protein of SARS-CoV-2, which plays a key role in the receptor recognition and cell membrane fusion process.

As used herein, the term “SARS-CoV-2 S1 protein,” refers to the S1 subunit of the SARS-CoV-2 spike protein.

As used herein, the term “subject” refers to any human or non-human mammal to which a composition in accordance with the invention may be administered, e.g., for experimental, diagnostic, prophylactic, and/or therapeutic purposes. In some embodiments, “subject” refers to a human or non-human mammal at any stage of development. In certain embodiments, the non-human mammal is, for example, a primate, monkey, rodent, mouse, rat, rabbit, monkey, dog, cat, sheep, pig, cattle, or sheep. In some embodiments, the animal is a transgenic or genetically-engineered mammal.

As used herein, the term “substantially” refers to the qualitative condition of exhibiting total or near-total extent or degree of a characteristic or property of interest (e.g., at least about 80%, 85%, 90%, 91%, 92%, 93%, 94%, 95%, 96%, 97%, 98%, 99%, or 99.5% in relation to a reference property of interest). One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The terms “treat” and “treatment” refer to the amelioration of one or more symptoms associated with a coronavirus infection; prevention or delay of the onset of one or more symptoms of a viral infection; and/or lessening of the severity or frequency of one or more symptoms of the infection.

### II. Compositions and Formulations

### A. Olive Component Composition

One aspect of the present application relates to an olive component (OC) composition having anti-viral activity. As used herein, the term “olive component (OC)” refers to chemical compounds, complexes or materials that are present in olives, the fruit of Olea europaea, or in products derived from olives, such as olive oil vegetation waters and the acidic hydrolysis products thereof. An OC may be obtained from a non-olive fruit or plant, or by chemical synthesis.

In some embodiments, the OC composition of the present application comprises one or more polyphenols. Polyphenols are a group of natural products occurring in tissues of all higher plants. Polyphenols are characterized by the presence of large multiples of phenol structural units. The number and characteristics of these phenol structures underly the unique physical, chemical, and biological (e.g., metabolic, toxic, and therapeutic) properties of particular members of the class. Polyphenols may be broadly classified as phenolic acids, flavonoids, stilbenes, and lignans. Of the various plants, the olive (Olea europaea L.) contains a large number of polyphenols. Exemplary polyphenol compounds in olives for use in the OC compositions of the present application include, but are not limited to, hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, including glycosylated, deglycosylated, phosphorylated and dephosphorylated forms thereof.

In some embodiments, the OC composition of the present application comprises hydroxytyrosol, and one or more polyphenols selected from the group consisting of tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, including glycosylated, deglycosylated, phosphorylated and dephosphorylated forms thereof.

In some embodiments, the OC composition of the present application comprises (1) one or more polyphenols selected from the group consisting of tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, including glycosylated, deglycosylated, phosphorylated and dephosphorylated forms thereof, and (2) hydroxytyrosol in an amount less than 0.5 wt %, 0.2 wt %, 0.1 wt %, 0.05 wt %, 0.02 wt %, 0.01 wt %, 0.005 wt %, 0.002 wt %, 0.001 wt %, 0.0005 wt %, 0.0002 wt %, or 0.0001 Wt %.

In some embodiments, the OC composition of the present application further comprises one or more non-phenolic compounds. Exemplary non-phenolic compounds in olives for use in the composition of the present application include, but are not limited to, D-fructose, glucofuranoside, glucopyranoside, dihydroxyacetone, malic acid, trihydroxybutyric acid, glucitol, glycerol, propanoic acid, xylonic acid, xylulose, xylitol, arabinose, ribose, deoxyribose, galactopyranoside, ketogluconic acid and galactofuranose.

In some embodiments, the OC composition of the present application comprises one or more compounds selected from the group consisting of hydroxytyrosol, tyrosol, oleuropein, vanillic acid, gallic acid, caffeic acid, syringic acid, ferrulic acid, ellagic acid, elenolic acid, oleanolic acid, linoleic acid, oleic acid, luteolin, kaemperol, quercetin, cathechin, lycopene, apigenin, rutin, β-carotene, 9-hexadecenoic acid, cholestan-3-ol, 2-methylene, 13-heptadecynl-ol, cis-13-eicosenoid acid, decanoic acid, 3-hydroxy, 4-methoxy-phenylacetic acid, 3,4-dihydroxybenzoic acid, gentisic acid, 1-heptatriacotanol, apigenin, azulene and astaxanthin.

In some embodiments, the OC composition of the present application comprises (1) hydroxytyrosol, (2) propanoic acid, and (3) one or more compounds selected from the group consisting of tyrosol, oleuropein, vanillic acid, gallic acid, caffeic acid, syringic acid, ferrulic acid, ellagic acid, elenolic acid, oleanolic acid, linoleic acid, oleic acid, luteolin, kaemperol, quercetin, cathechin, lycopene, apigenin, rutin, β-carotene, 9-hexadecenoic acid, cholestan-3-ol, 2-methylene, 13-heptadecynl-ol, cis-13-eicosenoid acid, decanoic acid, 3-hydroxy, 4-methoxy-phenylacetic acid, 3,4-dihydrooxybenzoic acid, gentisic acid, 1-heptatriacotanol, apigenin, azulene, astaxanthin, D-fructose, glucofuranoside, glucopyranoside, dihydroxyacetone, malic acid, trihydroxybutyric acid, glucitol, glycerol, propanoic acid, xylonic acid, xylulose, xylitol, arabinose, ribose, deoxyribose, galactopyranoside, ketogluconic acid and galactofuranose.

In some embodiments, the OC composition of the present application comprises (1) hydroxytyrosol, (2) propanoic acid, and (3) one or more compounds selected from the group consisting of tyrosol, oleuropein, vanillic acid, gallic acid, caffeic acid, syringic acid, ferrulic acid, ellagic acid, elenolic acid, oleanolic acid, linoleic acid, oleic acid, luteolin, kaemperol, quercetin, cathechin, lycopene, apigenin, rutin, β-carotene, 9-hexadecenoic acid, cholestan-3-ol, 2-methylene, 13-heptadecynl-ol, cis-13-eicosenoid acid, decanoic acid, 3-hydroxy, 4-methoxy-phenylacetic acid, 3,4-dihydrooxybenzoic acid, gentisic acid, 1-heptatriacotanol, apigenin, azulene, astaxanthin, malic acid, trihydroxybutyric acid, glucitol, glycerol, propanoic acid, xylonic acid, xylulose, xylitol, arabinose, ribose, deoxyribose, galactopyranoside, ketogluconic acid and galactofuranose, wherein the OC composition does not comprise D-fructose, glucofuranoside, glucopyranoside and/or dihydroxyacetone, or wherein the OC composition comprises D-fructose, glucofuranoside, glucopyranoside and/or dihydroxyacetone in a total amount of less than 0.1 wt %, 0.01 wt % or 0.001 wt %.

In some embodiments, the OC composition of the present application comprises (1) hydroxytyrosol; and (2) one or more compounds selected from the group consisting of tyrosol, oleuropein, vanillic acid, gallic acid, caffeic acid, syringic acid, ferrulic acid, ellagic acid, elenolic acid, oleanolic acid, linoleic acid, oleic acid, luteolin, kaemperol, quercetin, cathechin, lycopene, beta-carotene, apigenin, rutin, 9-hexadecenoic acid, cholestan-3-ol, 2-methylene, 13-heptadecynl-ol, cis-13-eicosenoid acid, decanoic acid, 3-hydroxy, 4-methoxy-phenylacetic acid, 3,4-dihydrooxybenzoic acid, gentisic acid, 1-heptatriacotanol, apigenin, azulene and astaxanthin; wherein the OC composition does not comprise beta-carotene and/or lycopene, or wherein the OC composition comprises beta-carotene and/or lycopene in an total amount of less than 1 wt %, 0.1 wt % or 0.01 wt %.

In some embodiments, the OC composition of the present application comprises (1) hydroxytyrosol; and (2) one or more compounds selected from the group consisting of tyrosol, oleuropein, vanillic acid, gallic acid, caffeic acid, syringic acid, ferrulic acid, ellagic acid, elenolic acid, oleanolic acid, linoleic acid, oleic acid, luteolin, kaemperol, quercetin, cathechin, apigenin, rutin, lycopene, β-carotene, 9-hexadecenoic acid, cholestan-3-ol, 2-methylene, 13-heptadecynl-ol, cis-13-eicosenoid acid, decanoic acid, 3-hydroxy, 4-methoxy-phenylacetic acid, 3,4-dihydrooxybenzoic acid, gentisic acid, 1-heptatriacotanol, apigenin, azulene and astaxanthin; wherein the OC composition does not comprise gallic acid, linoleic acid, oleic acid, and/or luteolin, or wherein the OC composition comprises gallic acid, linoleic acid, oleic acid and/or luteolin in a total amount of less than 1 wt %, 0.1 wt % or 0.01 wt %.

In some embodiments, the OC composition of the present application comprises an aqueous olive pulp extract (AOPE), a concentrated AOPE or dried powder of AOPE.

In some embodiments, the OC composition of the present application comprises 0.1-50 wt %, 0.1-0.2 wt %, 0.1-0.5 wt %, 0.1-1 wt %, 0.1-2 wt %, 0.1-5 wt %, 0.1-10 wt %, 0.1-20 wt %, 0.2-0.5 wt %, 0.2-1 wt %, 0.2-2 wt %, 0.2-5 wt %, 0.2-10 wt %, 0.2-20 wt %, 0.2-50 wt %, 0.5-1 wt %, 0.5-2 wt %, 0.5-5 wt %, 0.5-10 wt %, 0.5-20 wt %, 0.5-50 wt %, 1-2 wt %, 1-5 wt %, 1-10 wt %, 1-20 wt %, 1-50 wt %, 2-5 wt %, 2-10 wt %, 2-20 wt %, 2-50 wt %, 5-10 wt %, 5-20 wt %, 5-50 wt %, 10-20 wt %, 10-50 wt % or 20-50 wt % phenolic compounds. In some embodiments, the OC composition of the present application comprises about 0.5 wt %, about 2 wt %, about 3 wt %, about 9 wt % or about 12 wt % phenolic compounds.

In some embodiments, the phenolic compounds of the above-described OC composition comprise (1) hydroxytyrosol in the range of 10-70 wt %, 10-15 wt %, 10-20 wt %, 10-25 wt %, 10-30 wt %, 10-35 wt %, 10-40 wt %, 10-45 wt %, 10-50 wt %, 10-55 wt %, 10-60 wt %, 10-65 wt %, 15-20 wt %, 15-25 wt %, 15-30 wt %, 15-35 wt %, 15-40 wt %, 15-45 wt %, 15-50 wt %, 15-55 wt %, 15-60 wt %, 15-65 wt %, 15-70 wt %, 20-25 wt %, 20-30 wt %, 20-35 wt %, 20-40 wt %, 20-45 wt %, 20-50 wt %, 20-55 wt %, 20-60 wt %, 20-65 wt %, 20-70 wt %, 25-30 wt %, 25-35 wt %, 25-40 wt %, 25-45 wt %, 25-50 wt %, 25-55 wt %, 25-60 wt %, 25-65 wt %, 25-70 wt %, 30-35 wt %, 30-40 wt %, 30-45 wt %, 30-50 wt %, 30-55 wt %, 30-60 wt %, 30-65 wt %, 30-70 wt %, 35-40 wt %, 35-45 wt %, 35-50 wt %, 35-55 wt %, 35-60 wt %, 35-65 wt %, 35-70 wt %, 40-45 wt %, 40-50 wt %, 40-55 wt %, 40-60 wt %, 40-65 wt %, 40-70 wt %, 45-50 wt %, 45-55 wt %, 45-60 wt %, 45-65 wt %, 45-70 wt %, 50-55 wt %, 50-60 wt %, 50-65 wt %, 50-70 wt %, 55-60 wt %, 55-65 wt %, 55-70 wt %, 60-65 wt %, 60-70 wt % or 65-70 wt % of the phenolic compounds, and/or (2) oleuropein in the range of 1-20 wt %, 1-2 wt %, 1-5 wt %, 1-10 wt %, 1-15 wt %, 2-5 wt %, 2-10 wt %, 2-15 wt %, 2-20 wt %, 5-10 wt %, 5-15 wt %, 5-20 wt %, 10-15 wt %, 10-20 wt % or 15-20 wt % of the phenolic compounds; and (3) tyrosol in the range of 0.03-3 wt %, 0.03-0.1 wt %, 0.03-0.3 wt %, 0.03-1 wt %, 0.1-0.3 wt %, 0.1-1 wt %, 0.1-3 wt %, 0.3-1 wt %, 0.3-3 wt % or 1-3 wt % the phenolic compounds. In some embodiments, phenolic compounds of the above-described OC composition comprise about 35-45 wt % hydroxytyrosol, 5-10 wt % oleuopein and 0.1-0.5 wt % tyrosol.

In some embodiments, the OC composition of the present application comprises an aqueous olive pulp extract, a concentrated aqueous olive pulp extract, or a dried aqueous olive pulp extract. In some embodiments, the OC composition of the present application comprises an aqueous olive pulp extract treated with acid hydrolysis. In some embodiment, the OC composition of the present application comprises one or more of the compounds in the amount shown in Table 1.

TABLE 1
Components of the OC composition of the present application

| Compound | wt % |
|---|---|
| Hydroxytyrosol | 10-40 |
| Tyrosol | 0.1-0.6 |
| Oleuropein | 1-6 |
| Vanillic Acid | 2-8 |
| Gallic Acid | 5-10 |
| Caffeic acid | 3-7 |
| Syringic acid | 2-8 |
| Ferulic Acid | 5-12 |
| Ellagic acid | 1-7 |
| Elenolic acid | 0.1-3 |
| Oleanolic acid | 0.1-3 |
| Linoleic Acid | 2-8 |
| Oleic acid | 2-8 |
| Luteolin | 2-8 |
| Kaemperol | 2-8 |
| Quercetin | 2-8 |
| Cathechin | 1-7 |
| licopene | 1-5 |
| Apigenin | 0.1-5 |
| Rutin | 0.1-4 |
| Beta-carotene | 2-12 |

In some embodiment, the OC composition of the present application comprises one or more of the compounds in the amount shown in Table 2.

TABLE 2
Composition of the OC composition of the present application

| Compound | wt % |
|---|---|
| Hydroxytyrosol | 19.2 |
| Tyrosol | 0.38 |
| Oleuropein | 3 |
| Vanillic Acid | 3.8 |
| Gallic Acid | 8.12 |
| Caffeic acid | 5.2 |
| Syringic acid | 4.8 |
| Ferulic Acid | 8.4 |
| Ellagic acid | 3.5 |
| Elenolic acid | 2.1 |
| Oleanolic acid | 0.7 |
| Linoleic Acid | 5.28 |
| Oleic acid | 4.4 |
| Luteolin | 4.5 |
| Kaemperol | 4.26 |
| Quercetin | 3.46 |
| Cathechin | 3.5 |
| licopene | 3 |
| Apigenin | 2.3 |
| Rutin | 1.85 |
| Beta-carotene | 8.25 |

### B. Formulation Comprising the OC Composition

Another aspect of the present application relates to an anti-viral formulation comprising the OC composition of the present application. In some embodiments, the anti-viral formulation is a nutraceutical. In some embodiments, the anti-viral formulation is formulated as a moisturizer lotion or cream for topical application. In some embodiments, the anti-viral formulation is formulated as a dietary supplement. In some embodiments, the anti-viral formulation is formulated as an aerosol for inhalation. In some embodiments, the anti-viral formulation is a pharmaceutical composition comprising the OC composition of the present application and a pharmaceutically acceptable carrier.

In some embodiments, the anti-viral formulation comprise the OC composition of the present application in an amount in the range of 0.01-99.9 wt %, 0.01-90 wt %, 0.01-60 wt %, 0.01-30 wt %, 0.01-10 wt %, 0.01-3 wt %, 0.01-1 wt %, 0.01-0.3 wt %, 0.01-0.1 wt %, 0.01-0.03 wt %, 0.03-99.9 wt %, 0.03-90 wt %, 0.03-60 wt %, 0.03-30 wt %, 0.03-10 wt %, 0.03-3 wt %, 0.03-1 wt %, 0.03-0.3 wt %, 0.03-0.1 wt %, 0.1-99.9 wt %, 0.1-90 wt %, 0.1-60 wt %, 0.1-30 wt %, 0.1-10 wt %, 0.1-3 wt %, 0.1-1 wt %, 0.1-0.3 wt %, 0.3-99.9 wt %, 0.3-90 wt %, 0.3-60 wt %, 0.3-30 wt %, 0.3-10 wt %, 0.3-3 wt %, 0.3-1 wt %, 1-99.9 wt %, 1-90 wt %, 1-60 wt %, 1-30 wt %, 1-10 wt %, 1-3 wt %, 3-99.9 wt %, 3-90 wt %, 3-60 wt %, 3-30 wt %, 3-10 wt %, 10-99.9 wt %, 10-90 wt %, 10-60 wt %, 10-30 wt %, 30-99.9 wt %, 30-90 wt %, 30-60 wt %, 60-90 wt %, 60-99.9 wt % or 90-99.9 wt % of the anti-viral formulation.

In some embodiments, the anti-viral formulation of the present application is a moisturizer comprising the OC composition of the present application and one or more components selected from the group consisting of Aloe Barbadensis Leaf Juice, Olive Oil, Glycerin, Stearyl Alcohol, Glyceryl Stearate, PEG-100 Stearate, Simmondsia chinensis (Jojoba), Seed Oil, Sorbitan Olivate, Lauryl Laurate, Sorbitan Stearate, Glycol Distearate, Sodium Hyaluronate, Allantoin, Cetyl Palmitate, Sorbitan Palmitate, Cetearyl Olivate, Camellia sinensis Leaf Extract, Xanthan Gum, Phenoxyethanol, Capryl Glycol, Ethylhexylglycerin, Hexylene Glycol and Citric Acid.

In some embodiments, the anti-viral formulation of the present application comprises one or more ingredients in amounts described in Table 3. In some embodiments, the anti-viral formulation of the present application comprises all the ingredients in amounts described in Table 3.

TABLE 3

| Ingredients | Percentage Range |
|---|---|
| Water (Aqua) | QS to 100 |
| Aloe Barbadensis Leaf Juice | 5-15 |
| Olea Europaea (Olive) Fruit Oil | 5-15 |
| Olea Europaea (Olive) Fruit Juice | 1-5 |
| Glycerin | 1-5 |
| Stearyl Alcohol | 1-5 |
| Dimethicone | 1-5 |
| Cetearyl Alcohol | 1-5 |
| Glyceryl Stearate | 1-5 |
| PEG-100 Stearate | 1-5 |
| Simmondsia Chinensis (Jojoba) Seed Oil | 1-5 |
| Sorbitan Olivate | 1-5 |
| Lauryl Laurate | 1-5 |
| Sorbitan Stearate | 1-5 |
| Glycol Distearate | 1-5 |
| Sodium Hyaluronate | 1-5 |
| Allantoin | ≤1 |
| Cetyl Palmitate | ≤1 |
| Sorbitan Palmitate | ≤1 |
| Cetearyl Olivate | ≤1 |
| Camellia Sinensis Leaf Extract | ≤1 |
| Xanthan Gum | ≤1 |
| Sclerotium Gum | ≤1 |
| Phenoxyethanol | ≤1 |
| Caprylyl Glycol | ≤1 |
| Ethylhexylglycerin | ≤1 |
| Hexylene Glycol | ≤1 |
| Citric Acid | ≤1 |

Generally, for pharmaceutical use, the anti-viral formulation of the present invention is formulated as a pharmaceutical composition comprising the OC composition of the present application, at least one pharmaceutically acceptable carrier, and optionally one or more secondary pharmaceutically active compounds.

When an olive component contains an acidic group as well as a basic group, the compound can form internal salts, which can be present in the OC compositions and anti-viral formulations described herein. When an olive component contains a hydrogen-donating heteroatom (e.g., NH), salts are contemplated to cover isomers formed by transfer of said hydrogen atom to a basic group or atom within the molecule. Pharmaceutically acceptable salts of the olive component include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The pharmaceutically acceptable carrier includes, but is not limited to, diluents, binders, lubricants, disintegrators, fillers, pH modifying agents, preservatives, antioxidants, solubility enhancers, and coating compositions.

The pharmaceutical composition of the present invention can be prepared in a manner known per se, which usually involves mixing the OC composition according to the disclosure with one or more pharmaceutically acceptable carriers, and, if desired, in combination with other pharmaceutical active compounds when necessary under aseptic conditions. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. The pharmaceutical composition is typically made in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain from 1 to 2000 mg (e.g., 10, 25, 50, 100, 200, 300, 400, 500, 600, 700, 800, 900 or 1000 mg) per unit dosage.

The OC composition or the anti-viral formulation of the present application olive compounds can be administered by a variety of routes including the oral, ocular, rectal, transdermal, subcutaneous, intravenous, intramuscular or intranasal routes, depending mainly on the specific preparation used. The OC composition or the anti-viral formulation of the present application will generally be administered in an “effective amount”, by which is meant any amount of each olive compound that, upon suitable administration, is sufficient individually, collectively or synergistically to achieve the desired therapeutic or prophylactic effect in the subject to which it is administered.

The anti-viral formulations described herein can be formulated for modified or controlled release. Examples of controlled release dosage forms include extended-release dosage forms, delayed release dosage forms, pulsatile release dosage forms, and combinations thereof. Formulations with different drug release mechanisms described above can be combined in a final dosage form comprising single or multiple units. Examples of multiple units include, but are not limited to, multilayer tablets and capsules containing tablets, beads, or granules. An immediate release portion can be added to the extended-release system by means of either applying an immediate release layer on top of the extended-release core using a coating or compression process or in a multiple unit system such as a capsule containing extended and immediate release beads.

In one embodiment, the OC composition of the present application is formulated for topical administration. Suitable topical dosage forms include lotions, creams, ointments, and gels. A “gel” is a semisolid system containing a dispersion of the active agent, i.e., olive compounds in a liquid vehicle that is rendered semisolid by the action of a thickening agent or polymeric material dissolved or suspended in the liquid vehicle. The liquid can include a lipophilic component, an aqueous component or both. Some emulsions can be gels or otherwise include a gel component. Some gels, however, are not emulsions because they do not contain a homogenized blend of immiscible components. Methods for preparing lotions, creams, ointments, and gels are well known in the art.

The OC composition of the present application can be stored as a lyophilized powder under aseptic conditions and combined with a sterile aqueous solution prior to administration. The aqueous solution used to resuspend the OC composition can contain pharmaceutically acceptable auxiliary substances as required to approximate physical conditions, such as pH adjusting and buffering agents, tonicity adjusting agents and the like, as discussed above. Alternatively, the OC composition can be stored as a suspension, preferable an aqueous suspension, prior to administration.

### III. Methods of Use

Another aspect of the present application relates to a method of preventing, treating, or ameliorating symptoms of a viral infection with the OC composition or formulation of the present application. In some embodiments, the method comprises the step of administering to a subject in need of such treatment, an effective amount of the OC composition or formulation of the present application.

In some embodiments, the subject is infected with, or is at risk of being infected by, a coronavirus. In some embodiments, the subject is infected with, or is at risk of being infected by a human SARS CoV-2 isolate, such as Wuhan-Hu-1 (NC_045512.2) and any CoV-2 isolates comprising a genomic sequence set forth in GenBank Accession Nos., such as MT079851 In some embodiments, the viral infection caused by a.1, MT470137.1, MT121215.1, MT438728.1, MT470115.1, MT358641.1, MT449678.1, MT438742.1, LC529905.1, MT438756.1, MT438751.1, MT460090.1, MT449643.1, MT385425.1, MT019529.1, MT449638.1, MT374105.1, MT449644.1, MT385421.1, MT365031.1, MT385424.1, MT334529.1, MT466071.1, MT461669.1, MT449639.1, MT415321.1, MT385430.1, MT135041.1, MT470179.1, MT470167.1, MT470143.1, MT365029.1, MT114413.1, MT192772.1, MT135043.1, MT049951.1; human SARS CoV-1 isolates, such as SARS CoV.A022 (AY686863), SARSCoV.CUHK-W1 (AY278554), SARSCoV.GDO1 (AY278489), SARSCoV.HC.SZ.61.03 (AY515512), SARSCoV.SZ16 (AY304488), SARSCoV.Urbani (AY278741), SARSCoV.civet010 (AY572035), SARSCoV.MA.15 (DQ497008); bat SARS CoV isolates, such as BtSARS.HKU3.1 (DQ022305), BtSARS.HKU3.2 (DQ084199), BtSARS.HKU3.3 (DQ084200), BtSARS.Rml (DQ412043), BtCoV.279.2005 (DQ648857), BtSARS.Rf1 (DQ412042), BtCoV.273.2005 (DQ648856), BtSARS.Rp3 (DQ071615), SARS-CoV-2 Delta variants (B.1.617.2 and AY lineages), SARS-CoV-2 omicron variants (B.1.1.529), SARS-CoV-2 Alpha variants (B.1.1.7 and Q lineages) SARS-CoV-2 Beta variants (B.1.351 and descendent lineages), SARS-CoV-2 Gamma vairants (P.1 and descendent lineages), SARS-CoV-2 Epsilon variants (B.1.427 and B.1.429), SARS-CoV-2 Eta vairants (B.1.525), SARS-CoV-2 Iota variants (B.1.526), SARS-CoV-2 Kappa variants (B.1.617.1), SARS-CoV-2 variants 1.617.3, SARS-CoV-2 Mu variants (B.1.621, B.1.621.1) and SARS-CoV-2 Zeta variants (P.2), as well as any subtype, clade or sub-clade thereof, including any other subgroup 2b coronavirus now known (e.g., as can be found in the GenBank® Database) or later identified in the GenBank® Database.

In some embodiments, the subject is infected with, or is at risk of being infected by, influenza A virus including subtype H1N1, H3N2, H7N9, or H5N1, influenza B virus, influenza C virus, rotavirus A, rotavirus B, rotavirus C, rotavirus D, rotavirus E, human coronavirus, SARS coronavirus, MERS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), Dengue virus, Zika virus, chikungunya, Eastern equine encephalitis virus (EEEV), Western equine encephalitis virus (WEEV), Venezuelan equine encephalitis virus (VEEV), Ross River virus, Barmah Forest virus, yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV), Human T-lymphotropic virus Type I (HTLV-1), Friend spleen focus-forming virus (SFFV), Xenotropic MuLVRelated Virus (XMRV) or Zika virus.

For prophylactic or therapeutic applications, the OC composition or formulation of the present application be administered by any route, including but not limited to any of the various parenteral, gastrointestinal, inhalation, and topical (epicutaneous) routes of administration. Parenteral administration generally involves injections or infusions and includes, for example, intravenous, intraarterial, intratumoral, intracardiac, intramuscular, intravesicular (e.g., to the bladder), intracerebral, intracerebroventricular, intraosseous infusion, intravitreal, intaarticular, intrathecal, epidural, intradermal, subcutaneous, transdermal, and intraperitoneal administration. Gastrointestinal administration includes oral, buccal, sublingual and rectal administration. The route of administration may involve local or systemic delivery of the OC composition or formulation.

In certain embodiments, the OC composition of the present application are delivered transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

As a general proposition, a therapeutically effective amount of an olive compound composition administered will be in a weight range of about 1 ng/kg body weight/day to about 100 mg/kg body weight/day whether by one or more administrations. In some embodiments, the OC composition or formulation of the present application is administered in weight range from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In some embodiments, the OC composition or formulation of the present application is administered every 4, 6, 8, 12 or 24 hours for a period of 1, 2, 3, 4, 5, 6, 7, 8, 9 or 10 days. In some embodiments, the OC composition or formulation of the present application is administered every 1, 2, 3, 4, 5, 6 or 7 days for a period of 3, 7, 14, 21 or 28 days.

In some embodiments, the OC composition of the present application is formulated as a cream or lotion or moisturizer. The method comprises the step of applying the cream or lotion to a surface of a skin for a period of 1-60 min, 1-45 min, 1-30 min, 1-15 min, 1-10 min, 1-5 min, 5-60 min, 5-45 min, 5-30 min, 5-15 min, 5-10 min, 10-60 min, 10-45 min, 10-30 min, 10-15 min, 30-60 min, 30-45 min, 45-60 min.

Another aspect of the present application relates to a method for preventing viral transmission. The method comprises the step of treating a surface with an effective amount of an anti-viral composition comprising the OC composition of the present application for a desired period of time to inactive virus on the surface. In some embodiments, the anti-viral composition is formulated as a cream, lotion or moisturizer. In some embodiments, the anti-viral composition is formulated as a moisturizer with ingredients shown in Table 3.

Another aspect of the present application relates to a method for preventing viral infection or ameliorating a symptom of a viral infection in a subject. The method comprises the step of administering to the subject an effective amount of a dietary supplement comprising the OC composition of the present application.

Another aspect of the present application relates to a method for preventing viral infection or ameliorating a symptom of a viral infection in a subject. The method comprises the step of administering to the subject an effective amount of the OC composition of the present application, wherein the OC composition is formulized in an aerosol and wherein the aerosol is administered by inhalation.

Combination Therapy

In some embodiments, the method of the present application further comprises a step of administering to the subject one or more secondary active compounds. The one or more secondary active compounds may be administered before, after or concurrently with the OC composition or formulation of the present application. Examples of the secondary active compounds include, but are not limited to, other antiviral agents, analgesics, anti-inflammatory drugs, antipyretics, antidepressants, antiepileptics, antihistamines, antimigraine drugs, antimuscarinics, anxiolytics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

Examples of other antiviral agents include, but are not limited to, abacavir, acyclovir, acyclovir, adefovir, amantadine, amprenavir, ampligen, arbidol, atazanavir, atripla, balapiravir, BCX4430, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, and zidovudine.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Methods

Viruses and Cells.

SARS-CoV-2 (JPN/TY/WK-521 strain), variant strains of SARS-CoV-2, including UK variant strain (hCoV-19/Japan/QHN001/2020), Brazilian variant strain (hCoV-19/Japan/TY7-501/2021), South African variant strain (hCoV-19/Japan/TY8-612/2021), Delta variant strain (hCoV-19/Japan/TY11-927-P1/2021) were obtained from the National Institute of Infectious Diseases (Tokyo, Japan). The VeroE6/TMPRSS2 cell line, which was established in the National Institute of Infectious Diseases, was obtained from the Japanese Collection of Research Bioresources (Osaka, Japan, Cell No. JCRB1819). For passaging, VeroE6/TMPRSS2 cells were cultured in Dulbecco's Modified Eagle's minimal essential medium (DMEM; Nissui Pharmaceutical Co., Ltd., Tokyo, Japan) supplemented with 10% fetal bovine serum, 2 mM L-glutamine (FUJIFILM Wako Pure Chemical Co., Osaka, Japan), 0.15% NaHCO₃(FUJIFILM Wako Pure Chemical Co.), 2 μg/mL amphotericin B (Bristol-Myers Squibb Co., New York, NY, USA), 100 μg/mL kanamycin (Meiji Seika Pharma Co., Ltd., Tokyo, Japan), and 100 μg/mL G418 disulfate aqueous solution (Nacalai Tesque Inc., Kyoto, Japan). After inoculation with SARS-CoV-2, VeroE6/TMPRSS2 cells were cultured in viral growth medium (VGM) composed of DMEM supplemented with 1% fetal bovine serum, 20 mM L-glutamine, 0.15% NaHCO₃, 2 μg/mL amphotericin B, and 100 μg/mL of kanamycin for 3 days. Viral stocks were obtained from cell culture supernatants (VGM containing SARS-CoV-2) and stored at −80° C. [viral titers: 6.90 to 7.25 log₁₀ 50% tissue culture infective doses (TCID₅₀)/mL]. SARS-CoV-2 was handled in the biosafety level 3 facility.

Preparation of HIDROX® 12% Powder

Olives are crushed in water to form an olive slurry. The aqueous portion of the slurry (“vegetation water” or “olive juice”) is separated, collected and acidified with food grade citric acid to a specific pH range. The acid-treated olive juice is filtrated and freeze dried to produce a powder comprising about 12% phenolic compounds (HIDROX® freeze-dried powder 12%).

Preparation of HIDROX® Solution

In an exemplary method for preparing HIDROX® solution, 1 g of HIDROX® 12% (Oliphenol LLC., CA, USA) powder was dissolved in 10 mL of phosphate-buffer saline (PBS) and a centrifugation (850×g, 10 min) was performed. The aqueous layer collected was stored at −30° C. and used as the stock of 100 mg/mL HIDROX® solution. For the preparation of 3-Hydroxytyrosol (HT) solution, 1 g of 3-Hydroxytyrosol (Tokyo Chemical Industry Co., Ltd., Tokyo, Japan) was dissolved in 10 mL of PBS and the stock of 100 mg/mL HT solution was also stored at −30° C. Various concentrations of HIDROX® and HT solutions were prepared by diluting the stock solutions with PBS before using. In addition, OLIVENOL™ plus⁺ Healing Moisturizer (Oliphenol, LLC) containing 2%, 5%, or 10% HIDROX® was prepared as the HIDROX®-containing cream. The HIDROX®-free (0%) base ingredients of OLIVENOL™ plus⁺ Healing Moisturizer was used as the base control.

Western Blotting (WB)

In the experiment analyzing the impact of the test compound on the structure of viral particle, SARS-CoV-2 solution (7.25 log₁₀ TCID₅₀/mL) was mixed with 9 times the amount of HIDROX® solution or HT solution. In the experiment analyzing the impact of the test compound on viral proteins including spike (S) protein S1 subunit, S protein receptor-binding domain (RBD), S protein S2 subunit, and nucleocapsid (N) protein, these recombinant viral proteins were mixed with HIDROX® solution or HT solution. The product information of these recombinant proteins is shown in Table 4. The final concentration of HIDROX® and HT in the mixture was 0.90 mg/mL. As a diluent control, PBS was mixed with the viral solution or recombinant proteins. The final concentration of recombinant proteins in the mixture was approximately 10 μg/mL. In the experiment analyzing the interaction of HIDROX® or HT with carbohydrate chains expressed on S proteins, approximately 10 μg/mL of recombinant S proteins (S1 subunit, RBD, and S2 subunit) were incubated at 37° C. overnight with or without 6500 units/mL of PNGase F PRIME™ Glycosidase (N-Zyme Scientifics LLC., PA, USA). The glycosidase-treated or untreated proteins were mixed with PBS, HIDROX® solution, or HT solution. The final concentration of HIDROX® and HT in the mixtures was 0.90 mg/mL.

These mixtures were placed at 25° C. for 0 hr (no reaction time) or 24 hr and then mixed with sodium dodecyl sulfate (SDS)-buffer with 2-mercaptoethanol (FUJIFILM Wako Pure Chemical Co.). These samples were subjected to SDS-polyacrylamide gel electrophoresis (SDS-PAGE) and WB as previously described (Takeda Y et al., Viruses 2020, 12:699). S protein S1 subunit, S protein RBD, S protein S2 subunit, and N protein were detected using primary and secondary antibodies as shown in Table 4.

TABLE 4
Recombinant proteins and antibodies.

| Recombinant | Primary antibody | Secondary antibody |
|---|---|---|
| protein | for WB | for WB |
| SARS-CoV-2 (2019-nCoV) Spike | SARS-CoV-2/2019-nCoV | Mouse anti-rabbit IgG |
| S1-His Recombinant Protein | Spike Antibody, Rabbit PAb | peroxidase conjugate |
| [Catalog No. 40591-V08H]* | [Catalog No. 40591]* | [Catalog No. A1949]^{‡} |
| Recombinant 2019-nCoV Spike | SARS-CoV-2 Spike RBD | |
| Protein (RBD, mFc Tag) | Polyclonal Antibody | |
| [Catalog No. PKSR030500]^{†} | [Catalog No. E-AB-V1006]^{†} | |
| SARS-CoV-2 (2019-nCoV) Spike | SARS-CoV-2/2019-nCoV | |
| Protein (S2 ECD, His tag) | Spike/S2 Antibody, | |
| [Catalog No. 40590-V08B]* | Rabbit PAb | |
| | [Catalog No. 40590-T62]* | |
| SARS-CoV-2 (2019-nCoV) | SARS-CoV-2 (2019-nCoV) | |
| Nucleocapsid Protein (His tag) | Nucleoprotein/NP | |
| [Catalog No. 40588-V08B]* | Antibody, Rabbit Mab | |
| | [Catalog No. 40143-R019]* | |

*Purchased from Sino Biological Inc., Beijing, China
^{†}Purchased from Elabscience Biotechnology Inc., TX, USA
^{‡}Purchased from Sigma-Aldrich, Inc., Saint Louis, MO, USA

Real-Time RT-PCR

SARS-CoV-2 solution (7.25 log₁₀ TCID₅₀/mL) was mixed with 9 times the amount of HIDROX® solution or HT solution. The final concentration of HIDROX® and HT in the mixture was 0.90 mg/mL. As a diluent control, PBS was mixed with the viral solution. These mixtures were placed at 25° C. for 0 hr (no reaction time) or 24 hr and then RNA was extracted using ISOGEN-LS (Nippon Gene, Tokyo, Japan) according to the manufacturer protocol. Five-hundred ng of RNA thus obtained was reverse transcribed using FastGene cDNA Synthesis 5× ReadyMix OdT (NIPPON Genetics Co, Ltd., Tokyo, Japan). Then real-time RT-PCR was performed using EagleTaq Master Mix with ROX (F. Hoffmann-La Roche Ltd., Basel, Switzerland) with the primers and probe as described in Shirato K et al., Jpn. J. Infect. Dis 2020, 73:304-307. The real-time RT-PCR conditions were: 50° C. for 2 min; 95° C. for 10 min; 45 cycles of 95° C. for 15 sec and 50° C. for 1 min.

Test Solutions

1.0 g of HIDROX® 12% (Oliphenol LLC., CA, USA) was dissolved in 10 mL of phosphate-buffered saline (PBS) and centrifuged at 850×g for 10 min. The aqueous layer collected was stored at −30° C. as a 100 mg/mL HIDROX® stock solution. A 1.0 mg/mL of HIDROX® solution was prepared by dilution of the stock solutions with PBS. PBS was used as a control solution.

Evaluation of Direct Virucidal Activity of Test Solutions

A viral solution (VGM containing variant SARS-CoV-2) was mixed with nine volumes of 1.0 mg/mL of HIDROX® solution. The final concentrations of HIDROX® in the mixture was 0.9 mg/mL. As a diluent control, PBS was mixed with the viral solution. The mixtures were incubated at 25° C. for 3 or 24 h and then inoculated into the cells, and a tenfold serial dilution was performed. After a 3 d incubation of the cells at 37° C., a cytopathic effect was observed, and the viral titer (log₁₀ TCID₅₀/mL) was calculated using the Behrens-Kärber method. The detection limit of the viral titer in each test solution group was determined based on the cytotoxicity of each test solution. The detection limits of the viral titers in the groups treated with PBS or HIDROX® solution were set to 1.25 and 2.25 log₁₀ TCID₅₀/mL, respectively.

Statistical Analysis

Student's t test was performed to determine statistically significant differences between a control group (e.g, PBS) and each test sample group (e.g., HIDROX®-treated). P values of less than 0.05 were considered statistically significant.

### Example 2: Time- and Concentration-Dependent Virucidal Activity of HIDROX® Solution Against SARS-CoV-2

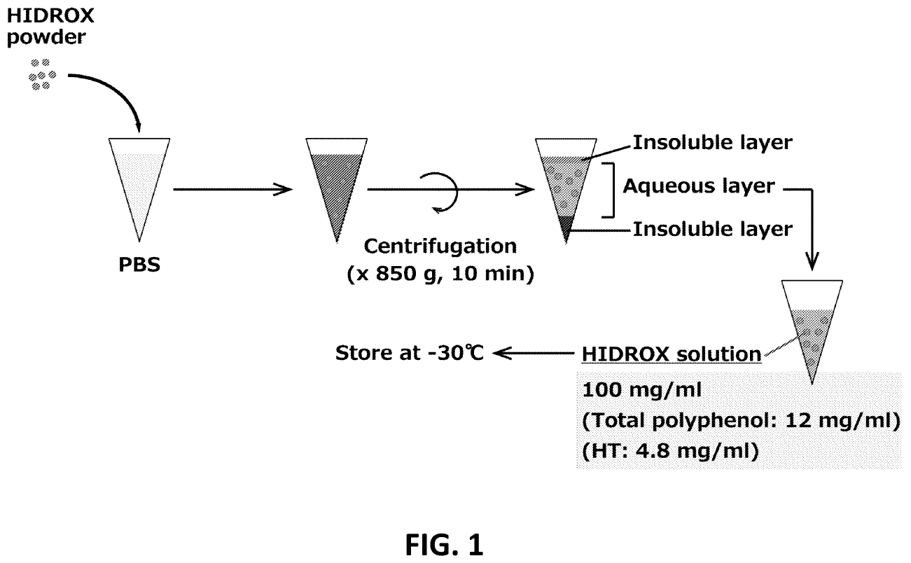

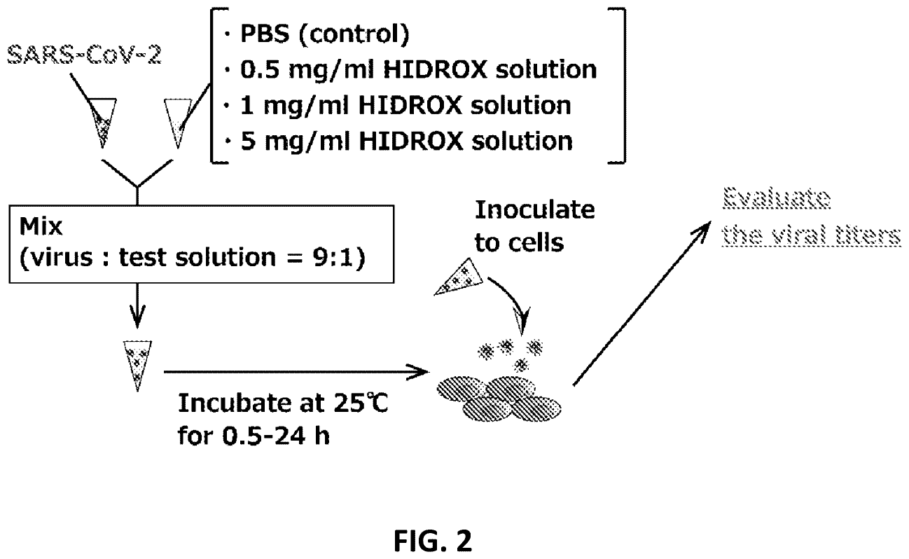

A HIDROX® solution was prepared in accordance with the schematic illustration depicted in FIG. 1 and the specific method steps outlined in Example 1. The virucidal activity of the HIDROX® solution against SARS-CoV-2 (JPN/TY/WK-521 strain) was evaluated in accordance with the experimental protocol depicted in FIG. 2 and the corresponding method steps below. Briefly, a SARS-CoV-2 solution (7.25 log₁₀ TCID₅₀/mL) was mixed with 9 times the amount of HIDROX® solution or HT solution. The final concentrations of HIDROX® and HT in the mixture were 0.45-11.25 mg/mL and 0.05-0.90 mg/mL, respectively. As a diluent control, PBS was mixed with the viral solution. These mixtures were placed at 25° C. for 5 min-24 hr and then inoculated into cells, and a tenfold serial dilution was performed. After incubation for 3 days, a cytopathic effect caused by inoculated SARS-CoV-2 was observed and the viral titer (log₁₀ TCID₅₀/mL) was calculated using the Behrens-Kärber method (Kärber, G., Naunyn-Schmiedebergs Arch. Exp. Pathol. Pharmakol. 1931,162, 480-483; doi:10.1007/BF01863914).

The detection limits for viral titers in each test group were determined based on the cytotoxic concentration of each test solution. The detection limit was set higher in the group treated with a solution of higher cytotoxicity. PBS, 0.45 mg/mL HIDROX®, and 0.05 mg/mL HT solutions did not show any cytotoxicity, and the detection limit of the viral titer in the groups treated by these solutions was set to 1.25 log₁₀ TCID₅₀/mL, according to the viral titer calculation. In contrast, >0.90 mg/mL HIDROX® and 0.90 mg/mL HT solutions demonstrated cytotoxicity. The detection limits in the groups treated with 0.90, 4.50, 5.63 mg/mL HIDROX®, and 0.90 mg/mL HT solutions were set to 2.25 log₁₀ TCID₅₀/mL and the detection limit in the group treated by 11.25 mg/mL HIDROX® solution was set to 3.25 log₁₀ TCID₅₀/mL, respectively.

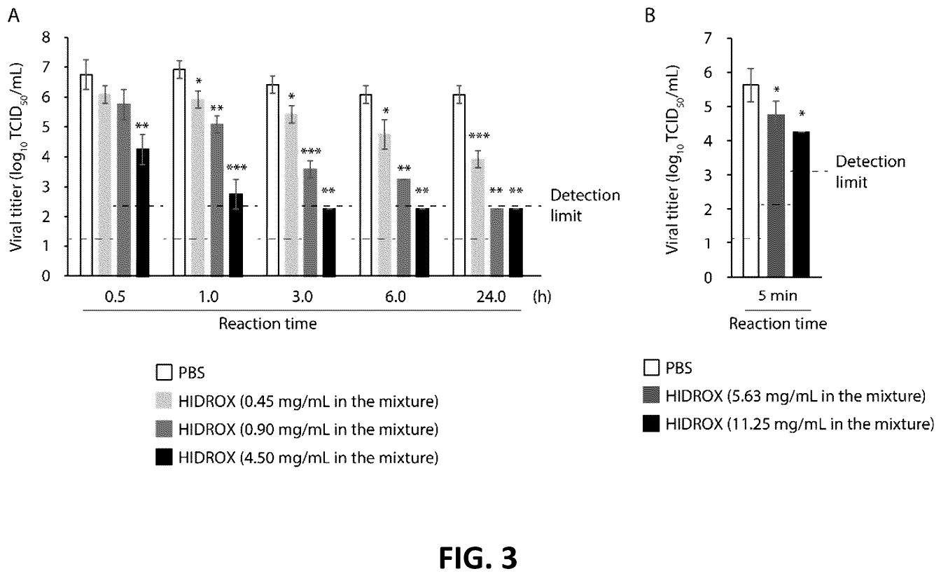

FIG. 3, panels A and B show the time- and concentration-dependent virucidal activities of HIDROX® against SARS-CoV-2. In these experiments, SARS-CoV-2 was mixed with HIDROX® such that the final concentrations of HIDROX® in each mixture were 0.45, 0.90, and 4.50 mg/mL (FIG. 1, panel A) or 5.63 and 11.25 mg/mL (FIG. 1, panel B). As a diluent control, PBS was mixed with the viral suspension. The mixtures were incubated at 25° C. for 0.5-24 h (Panel A) or 5 min (Panel B) and then the viral titers were evaluated.

As shown in FIG. 3, panel A, the HIDROX® solutions clearly showed time- and concentration-dependent SARS-CoV-2-inactivating activity. The 4.50 mg/mL of HIDROX® solution inactivated 99.68% of virus (2.50 log₁₀ TCID₅₀/mL reduction of viral titer) in 0.5 hr reaction time. The 0.90 mg/mL and 0.45 mg/mL of HIDROX® solutions inactivated 98.53% and 90.00% of virus (1.83 log₁₀ TCID₅₀/mL and 1.00 log₁₀ TCID₅₀/mL reduction) in 1.0 hr reaction time, respectively (FIG. 3, panel A). In addition, the 5.63 mg/mL and 11.25 mg/mL of HIDROX® solutions inactivated 86.67% and 95.78% of virus (0.88 log₁₀ TCID₅₀/mL and 1.38 log₁₀ TCID₅₀/mL reduction) in 5 min reaction time, respectively (FIG. 1, panel B). In FIG. 3, the results are presented as the mean±SD, where n=3-4 per group. Student's t-test was performed to evaluate the statistically significant difference between the PBS and HIDROX® groups; * p<0.05; ** p<0.01; *** p<0.001.

### Example 3. Time-Dependent Virucidal Activity of HIDROX® Solution Against Variant SARS-CoV-2 Strains

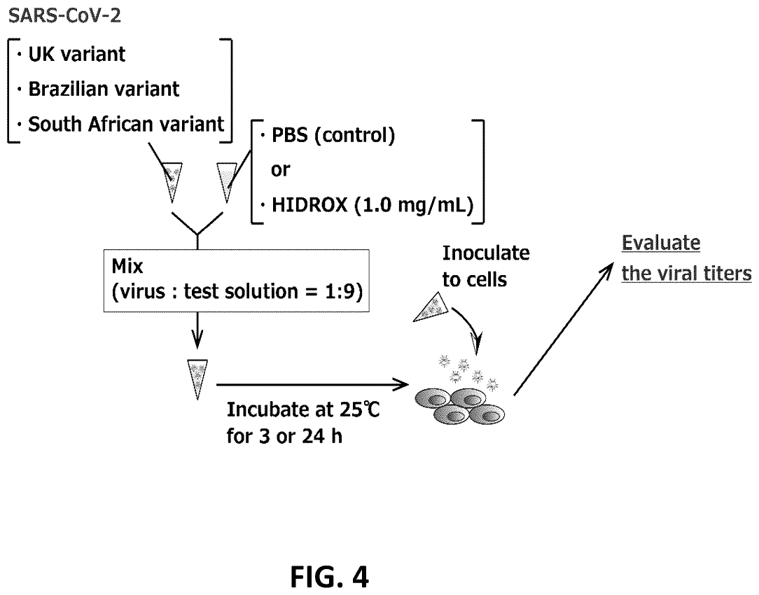

To extend the HIDROX® virucidal analysis to other SARS-CoV-2 variant strains, time-dependent virucidal activity of HIDROX® solution against four variant SARS-CoV-2 strains were determined according to the experimental protocol depicted in FIG. 4.

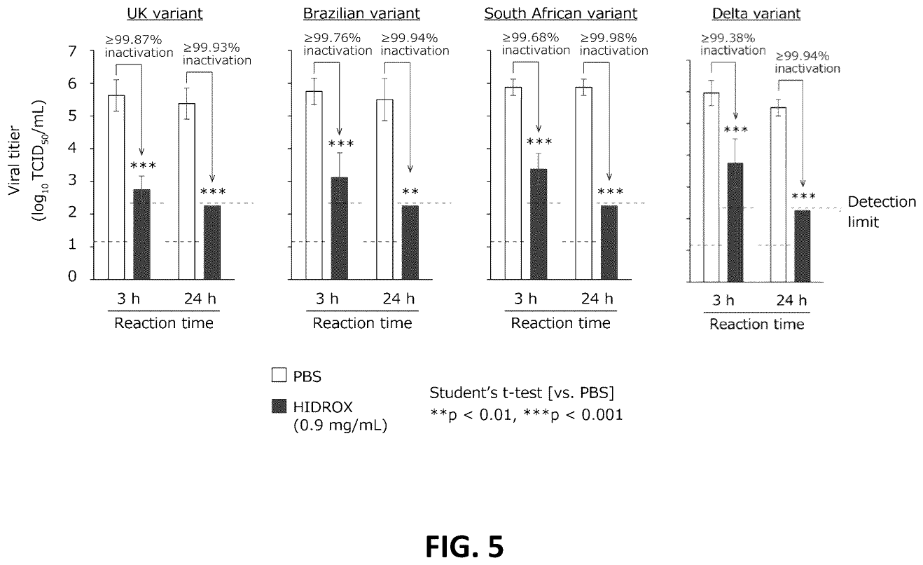

As shown in FIG. 5, HIDROX® was found to inactivate all four different SARS-CoV-2 variants. At a concentration of 0.9 mg/mL, HIDROX® inactivated >99% all the variant strains of SARS-CoV-2 in 3 h and inactivated >99.9% of the variant strains of SARS-CoV-2 in 24 h. The viral titers in the HIDROX® groups were below the detection limit.

### Example 4. Comparison of Virucidal Activity of HIDROX® Solution and HT Solution Against SARS-CoV-2

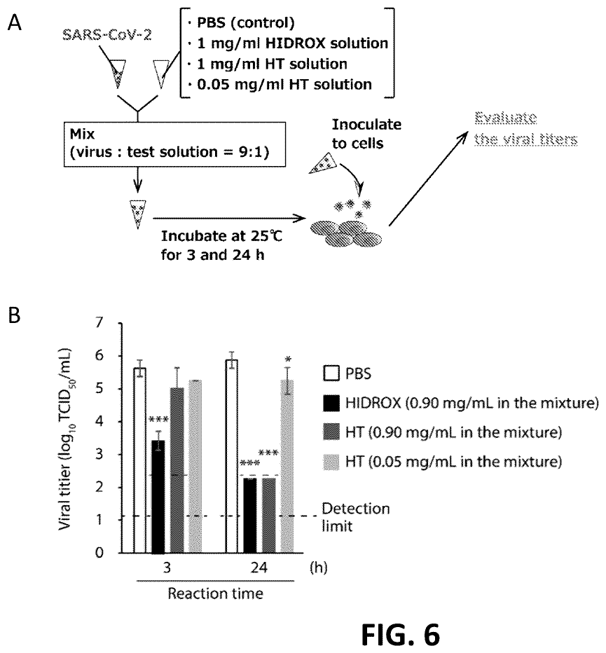

Next, the virucidal activities of HIDROX® solution and HT solution against SARS-CoV-2 were compared. In this experiment, FIG. 6, panel A depicts the experimental protocol for evaluating the comparative virucidal activities of HIDROX® and HT against SARS-CoV-2. Briefly, SARS-CoV-2 solutions (7.25 log₁₀ TCID₅₀/mL) were mixed with 9 times the amount of HIDROX® solution or HT solution and incubated at 25° C. for 3 or 24 hr. The final concentration of HIDROX® in the mixture was 0.90 mg/mL; the final concentration of HT in the mixtures were 0.90 mg/mL and 0.05 mg/mL. PBS was mixed with the viral suspension as the diluent control. Following the incubations at 25° C. for 3 or 24 hr, the mixtures were inoculated to cells and incubated for 3 days. At this point, a cytopathic caused by inoculated SARS-CoV-2 was observed and the viral titers (log₁₀ TCID₅₀/mL) in each group were calculated using the Behrens-Kärber method. The detection limit for the viral titer in each test group was determined based on the cytotoxic concentration of each test solution.

FIG. 6, panel B shows the results of the experiment depicted in panel A. In panel B, the detection limits of viral titer are 1.25 log₁₀ 50% tissue culture infective dose (TCID₅₀)/mL in the PBS and HT (0.05 mg/mL) groups and 2.25 log₁₀ TCID₅₀/mL in the HIDROX® (0.9 mg/mL) and HT (0.90 mg/mL) groups. Results are indicated as mean±SD (n=4 per group). Student's t-test was performed to evaluate the statistically significant difference between the PBS group and each test group; * p<0.05; *** p<0.001.

In addition to the same concentration (0.90 mg/mL) of HIDROX® and HT in the solutions, a 0.05 mg/ml of HT solution corresponding to the concentration of HT contained in the 0.90 mg/mL HIDROX® solution was tested. Following a 3 hr reaction time, the 0.90 mg/mL HIDROX® solution caused significant virucidal activity, while the 0.90 mg/mL and 0.05 mg/mL HT solutions did not. While both of 0.90 mg/mL of HIDROX® and HT solutions inactivated >99.98% of virus (>3.63 log₁₀ TCID₅₀/mL reduction; the viral titer was below the detection limit) in 24 hr reaction time, the virucidal activity of 0.05 mg/mL of HT solution was limited (0.63 log₁₀ TCID₅₀/mL reduction) (FIG. 6, panel B). These results indicate that the SARS-CoV-2-inactivating activity of HIDROX® is more potent than that of pure HT.

### Example 5. Virucidal Activity of HIDROX® in SARS-CoV-2 Infected Cells at Various Concentrations

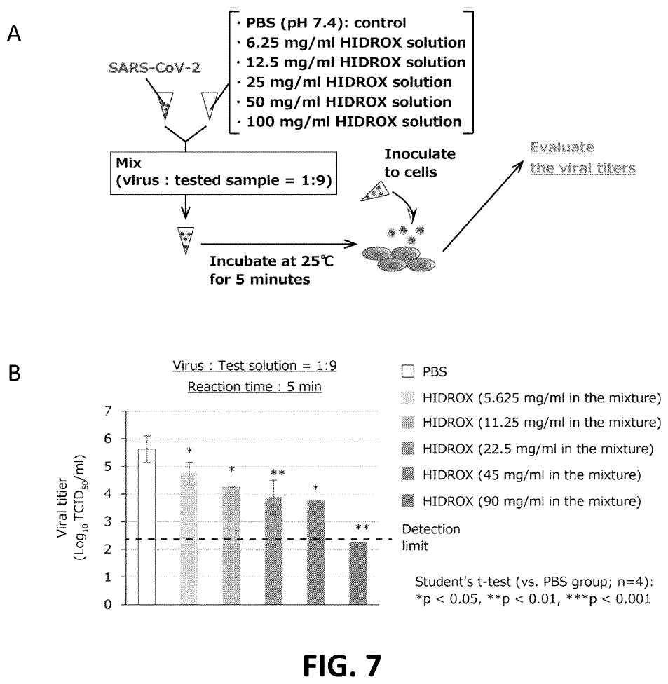

Virucidal activity of HIDROX® in SARS-CoV-2 infected cells at various concentrations was evaluated. FIG. 7, panel A shows an experimental protocol for examining the direct virucidal activity of HIDROX® against SARS-CoV-2 at various concentrations (6.25-100 mg/ml). FIG. 7, panel B shows the virucidal activity of HIDROX® at various concentrations in accordance with the schematic diagram in FIG. 7.

### Example 6. Inhibitory Effect of HIDROX® on SARS-CoV-2 Proliferation

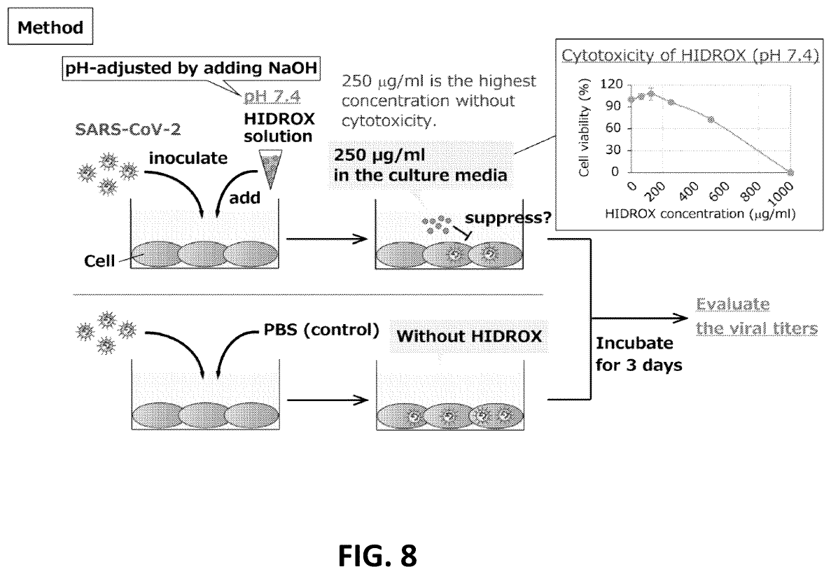

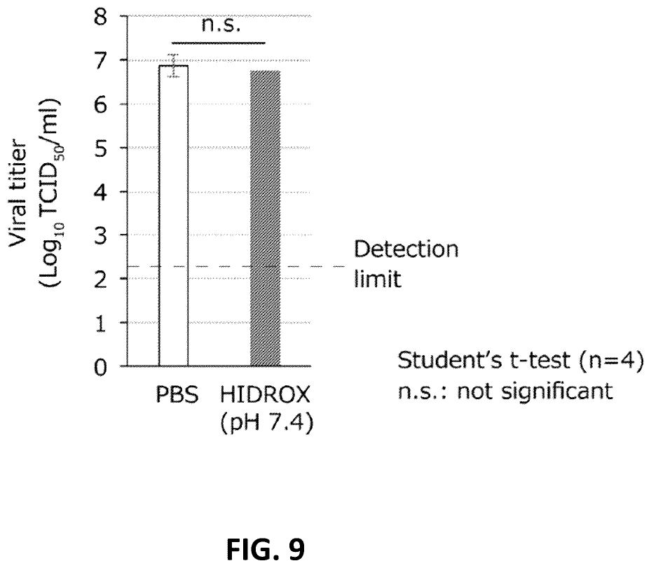

Inhibitory effect of HIDROX® on SARS-CoV-2 proliferation was evaluated. FIG. 8 shows an experimental protocol for evaluating the inhibitory effect of HIDROX® (pH 7.4) on virus proliferation in host cells. FIG. 9 shows the results of the experiment described in FIG. 8.

### Example 7. Virucidal Activity of HIDROX® in SARS-CoV-2 Infected Cells at Low Concentrations

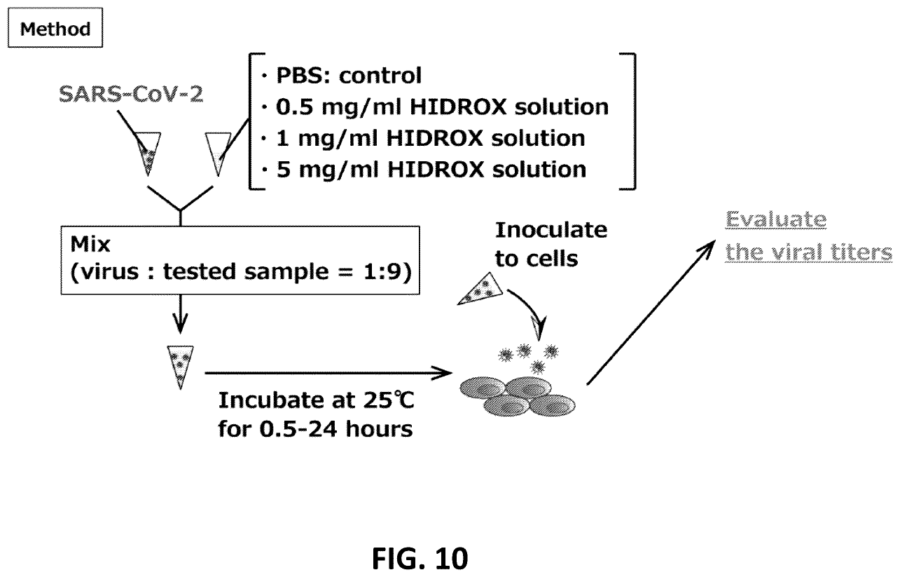

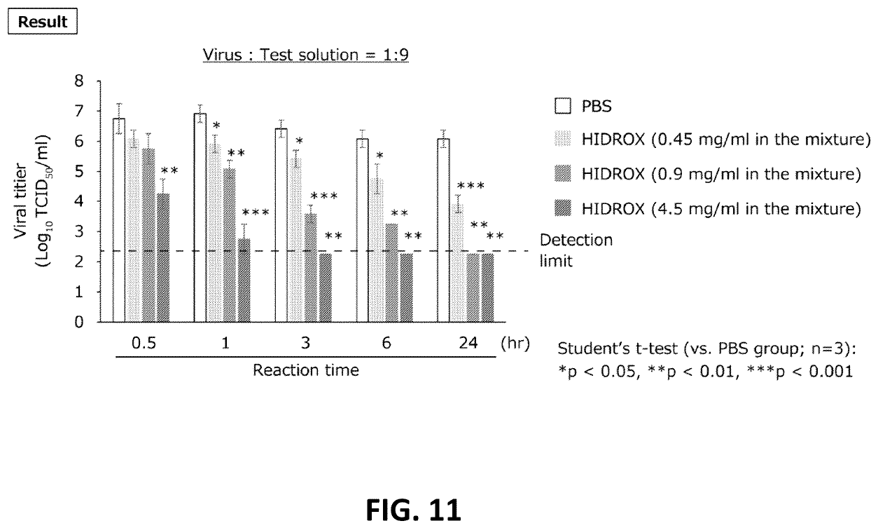

Virucidal activity of HIDROX® at low concentrations was evaluated in SARS-CoV-2 infected cells. FIG. 10 shows an experimental protocol for examining the direct virucidal activity of HIDROX® against SARS-CoV-2 at low concentrations (0.5-5.0 mg/ml). FIG. 11 shows the results of the experiment described in FIG. 10.

### Example 8. Cytotoxicity of HT Compared to HIDROX®

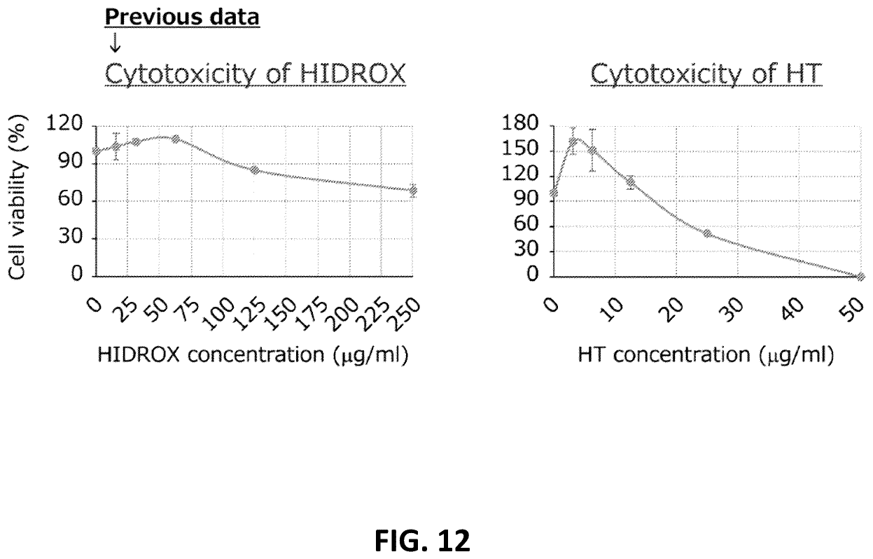

The cytotoxicity of HT and HIDROX® was evaluated. FIG. 12 shows the cytotoxicity of HT (0-50 μg/ml) compared to HIDROX® (0-250 μg/ml).

### Example 9. Comparison of the Virucidal Activities of HIDROX® and HT Against SARS-CoV-2

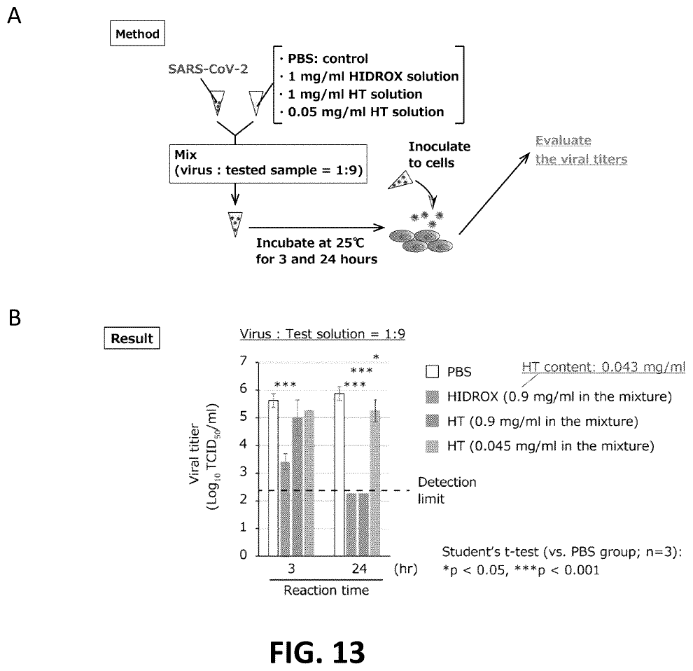

FIG. 13, panel A shows an experimental protocol for comparing the virucidal activities of HIDROX® and HT against SARS-CoV-2 at 1.0 mg/ml and 0.05 mg/ml. FIG. 13, panel B shows the result of the experiment described in panel A.

### Example 10. Time- and Concentration-Dependent Virucidal Activity of HIDROX®-Containing Cream Against SARS-CoV-2

The virucidal activity of HIDROX®-containing cream, which is assumed to be used for topical application like a hand cream, against SARS-CoV-2 was evaluated. Briefly, 20 mg of test cream was applied on 2.25 cm² (1.5 cm×1.5 cm) of polyethylene terephthalate film (AS ONE Co., Ltd., Osaka, Japan). The lid of 12 well plate (Nunc, Rochester, NY, USA) was turned over and 5.25 log₁₀ TCID₅₀/60 ml of SARS-CoV-2 solution was dropped to the inside of well-lid. The viral solution was covered by the cream-coated film. In this setting, the cream was in contact with the viral solution. The 12 well plate was placed for 10 min-6 hr at 25° C. After the predetermined reaction times, the viral solution was recovered. This viral solution was inoculated into cells and tenfold serial dilutions were prepared. After incubation for 1 hr at 37° C., the cell culture medium containing virus was removed and new VGM was added. After incubation for 3 days at 37° C., the viral titer (log₁₀ TCID₅₀/mL) was calculated.

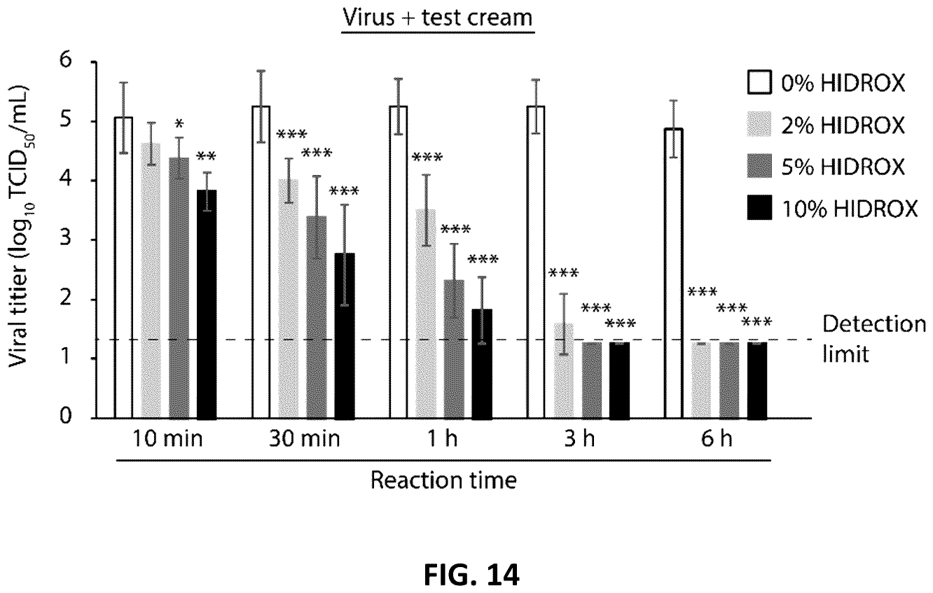

Upon application of 20 mg of HIDROX®-containing cream to 2.25 cm² of film clearly showed time- and concentration-dependent SARS-CoV-2-inactivating activity (FIG. 14). The 10% and 5% HIDROX®-containing creams inactivated 94.38% and 79.47% of virus (1.25 log₁₀ TCID₅₀/mL and 0.69 log₁₀ TCID₅₀/mL reduction of viral titer) in 10 min reaction time, respectively. The 2% HIDROX®-containing cream also inactivated 94.38% of virus (1.25 log₁₀ TCID₅₀/mL reduction of viral titer) in 30 min reaction time (FIG. 14).

### Example 11. Impact of HIDROX® and HT on SARS-CoV-2 Structural Proteins

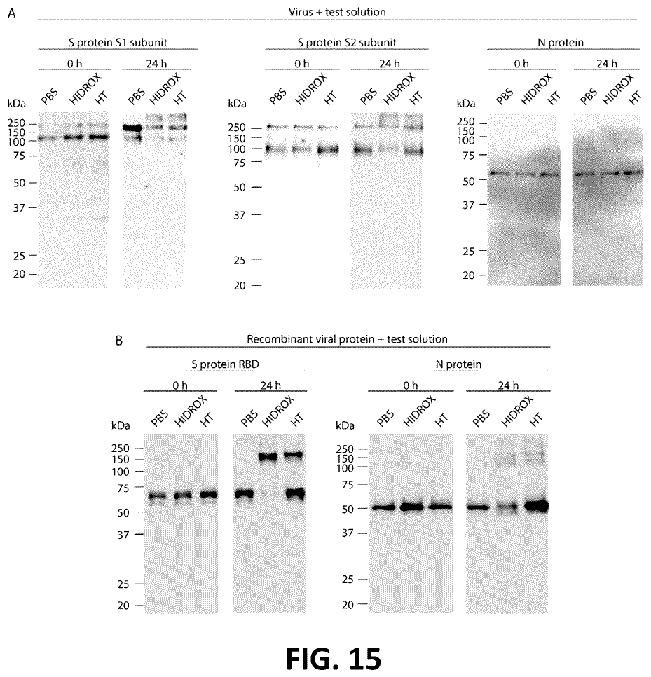

To evaluate the impact of HIDROX® and HT on SARS-CoV-2 structural proteins, the S protein S1 subunit, S protein S2 subunit, and N protein expressing on virus particles treated by PBS (diluent control), HIDROX®, and HT were analyzed by WB. The results of WB to detect S protein S1 subunit and S2 subunit on PBS treated-viruses showed two specific bands with different molecular weights; one was around 240 kDa and the other was around 120 kDa. It is presumed that the higher molecular weight band was the full-length S protein and the lower molecular weight band was the cleaved S protein, corresponding S1 or S2 subunits. While HIDROX®- and HT-treated viruses also showed these two bands in 0 hr (no reaction time), the intensity of these bands became weaker and the other bands or ladder with >250 kDa was appeared in 24 hr reaction time (FIG. 15, panel A, left and middle).

In the WB to detect N protein, there was no difference in the intensity of specific band among PBS-, HIDROX®-, and HT-treated viruses in both of 0 hr and 24 hr reaction times (FIG. 15, panel A, right). Next, to evaluate the impact of HIDROX® and HT on RBD located on S protein S1 subunit, the WB to detect RBD was performed. While the main band with ˜65 kDa was observed among all of PBS-, HIDROX®, and HT-treated recombinant proteins in 0 hr, this band was almost disappeared and the strong band with >150 kDa was appeared by HIDROX®-treatment in 24 hr reaction time. On the other hand, both of the two bands with ˜65 kDa and >150 kDa were detected in HT-treated protein in 24 hr reaction time (FIG. 15, panel B, left).

To evaluate the direct impact of HIDROX® and HT on N protein, the recombinant N protein was mixed with PBS, HIDROX®, or HT solution. The intensity of specific band with ˜50 kDa was slightly weaker in HIDROX®-treated protein than that in PBS-treated protein in 24 hr reaction time, but such a decrease of the intensity was not observed in HT-treated protein. In addition, several bands with >100 kDa were appeared by both of HIDROX®- and HT-treatment (FIG. 15, panel B, right). These results indicate that HIDROX® and HT induce some changes that affect the molecular weight for viral structural proteins, especially S proteins. In addition, these results also suggest that such a mode of action of HIDROX® is stronger than that of HT.

### Example 12. Interaction of HIDROX® or HT with Carbohydrate Chains Expressed on S Proteins

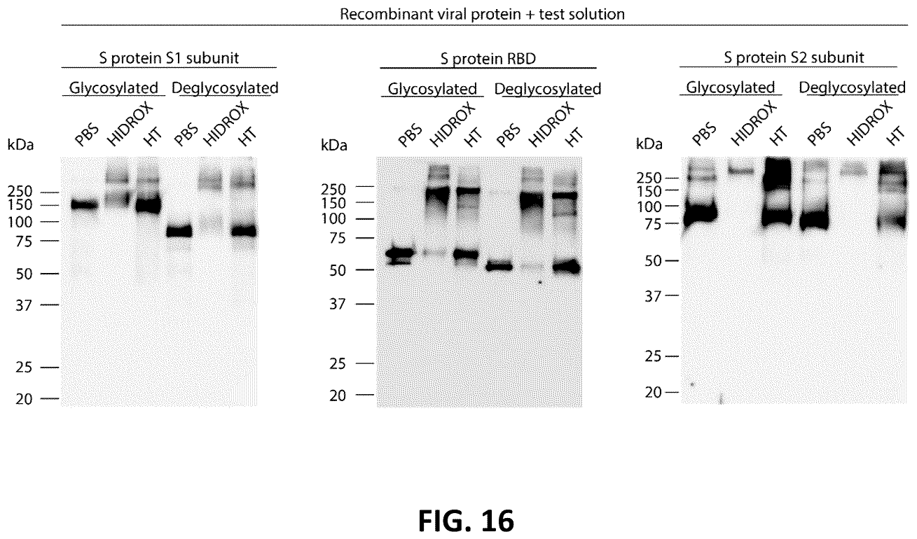

Since S protein of SARS-CoV-2 is highly glycosylated by post-translational modification. The interaction of HIDROX® and HT with carbohydrate chains attached to S protein was evaluated. The recombinant S protein S1 subunit, RBD, and S2 subunit which were non-treated (glycosylated) or treated by glycosidase (deglycosylated) beforehand were mixed with PBS, HIDROX®, or HT solution. After 24 hr reaction time WB was performed. In the WB to detect S1 subunit, the specific bands with ˜150 kDa and ˜75 kDa were detected in glycosylated- and deglycosylated-proteins treated by PBS, respectively. However, this deglycosylated-band with ˜75 kDa were disappeared by HIDROX®-treatment. In addition, the other bands with >250 kDa were appeared by HIDROX®- and HT-treatments in both of glycosylated- and deglycosylated-proteins (FIG. 16, left). In the WB to detect RBD, the molecular weight of the specific band observed in PBS-treated protein was slightly reduced by deglycosylation. Both of these glycosylated- and deglycosylated-bands were almost disappeared by HIDROX®-treatment, and the other bands with >150 kDa were appeared by both of HIDROX®- and HT-treatments (FIG. 16, middle). In the WB to detect S2 subunit, the molecular weight of the main specific band observed in PBS-treated protein was slightly reduced by deglycosylation. In addition, some weak bands with >˜250 kDa were also observed in PBS-treated proteins. However, both of glycosylated- and deglycosylated-main bands were disappeared by HIDROX®-treatment. On the other hand, the bands or ladder with >˜250 kDa remained (or appeared) in HIDROX®- and HT-treated proteins (FIG. 16, right). These results indicate that the site of action of HIDROX® and HT on S proteins was not carbohydrate chains.

### Example 13. Impact of HIDROX® and HT on SARS-CoV-2 Genome

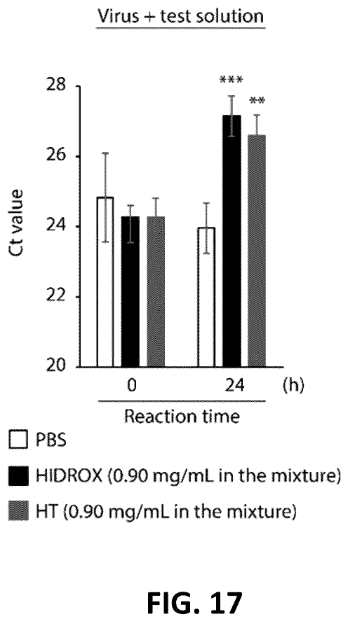

To evaluate the impact of HIDROX® and HT on SARS-CoV-2 genome, the viral RNA was extracted from PBS-, HIDROX®-, and HT-treated viruses and real-time RT-PCR was performed. While the Ct values were similar among all treatment groups in 0 hr (no reaction time), these values were 3.19 and 2.63 higher in HIDROX® and HT groups than that of PBS group in 24 hr reaction time, respectively. This increase of Ct values meant that the amount of viral RNA was 9.13 and 6.17 times lower in HIDROX® and HT groups than that of PBS group, respectively (FIG. 17). This result indicates that HIDROX® and HT disrupt viral genome.

### Example 14. Cytotoxicity of HT Compared to HIDROX® in MDCK Cells

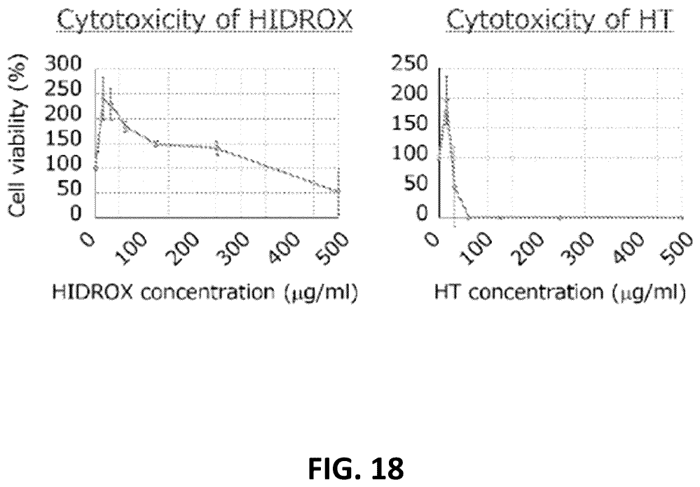

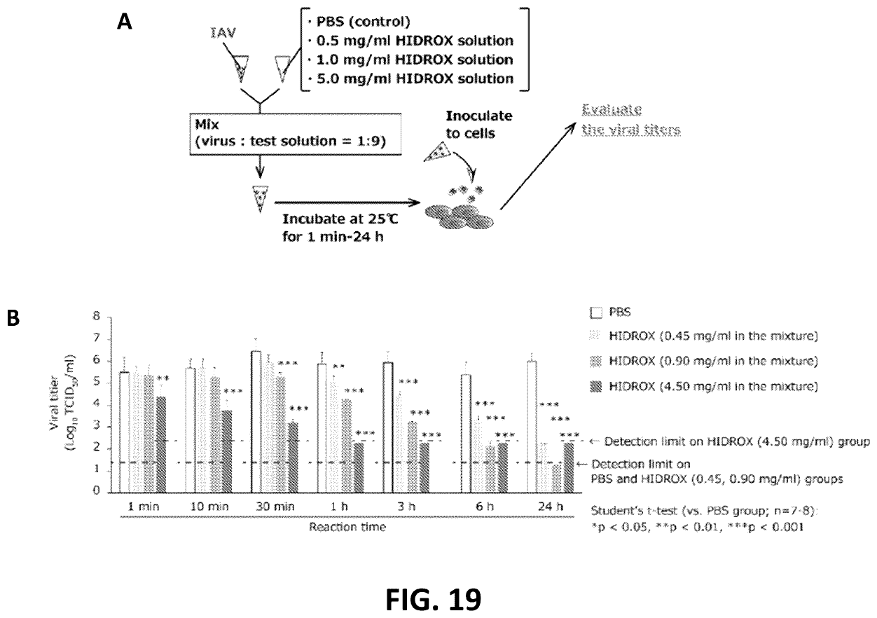

FIG. 18 shows the cytotoxicity of HT (0-500 μg/ml) compared to HIDROX® (0-500 μg/ml) in MDCK cells, the host cells for influenza (H1N1) in the experiment described in FIG. 19.

### Example 15. Virucidal Activity of HIDROX® Against Influenza-Infected MDCK Cells

Virucidal activity of HIDROX® was evaluated in influenza virus-infected MDCK cells. FIG. 19, panel A shows an experimental protocol for evaluating the virucidal activity of HIDROX® against influenza-infected MDCK cells at various different concentrations and incubation periods. FIG. 19, panel B shows the result of the experiment described in panel A.

### Example 16. Analysis of the OLIVENOL™ Dietary Supplement

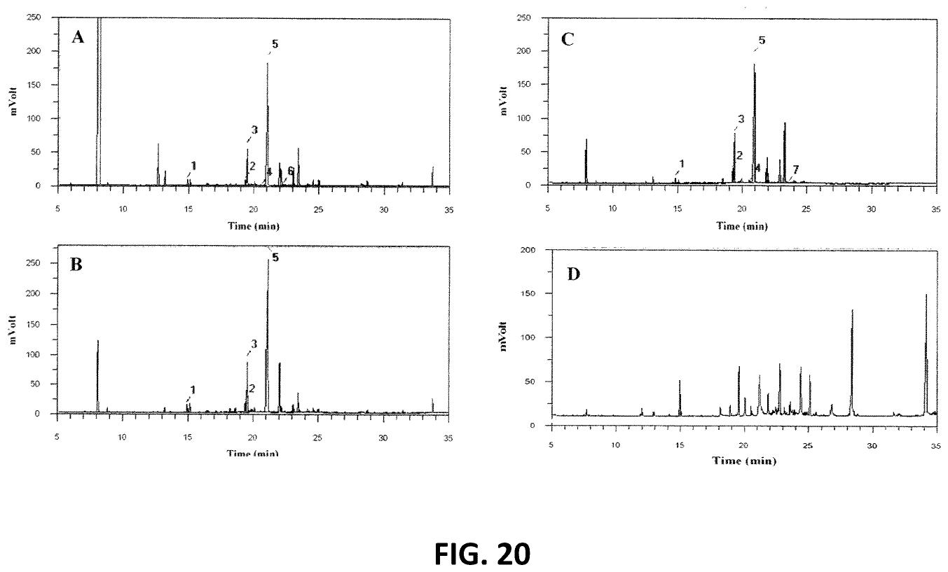

OLIVENOL™ is a dietary supplement. The amount of phenolic compounds in the OLIVENOL™ and two different batches of vegetation water from olive oil processing (CR43 and CR21) are shown in Table 5. Gas chromatographic profiles in the time range 15-25 min, generally attributable to simple phenols, have shown that OLIVENOL™ composition is qualitatively similar to that of the phenolic fraction of extra-virgin olive oil (FIG. 20), notwithstanding the quantitative contents being different. In particular, the concentrations of tyrosol and hydroxytyrosol in OLIVENOL™ are about 50 and 30-fold higher than that present in the extra-virgin olive oil studied.

TABLE 5
Phenolic compounds in the OLIVENOL ™ dietary supplement and in the aqueous phase
of vegetation waters (OVW) from olive oil processing batch CR43 and CR21. The analyzed
lot of OLIVENOL ™ was obtained from the aqueous phase of vegetation waters
from olive oil processing batch CR43. Data is expressed as the mean (n = 4) ± standard deviation.

| | Olivenol ™ | CR43 | CR21 |
|---|---|---|---|
| pH | 4.05 | 4.35 | 4.27 |
| Total phenol content^{a} (mg/ml) | 7.41 ± 0.90 | 6.91 ± 0.97 | 8.81 ± 0.31 |
| Dry weight (mg/ml) | 83.3 ± 8.9 | 50.0 ± 1.0 | 60.0 ± 3.0 |

Phenolic compounds content (mg/ml)

| | | | |
|---|---|---|---|
| Tyrosol | 0.26 ± 0.02 | 0.27 ± 0.03 | 0.21 ± 0.02 |
| Vanillic acid | 0.41 ± 0.04 | 0.65 ± 0.03 | 0.86 ± 0.14 |
| Hydroxytyrosol | 2.65 ± 0.40 | 1.91 ± 0.23 | 1.86 ± 0.25 |
| 3-hydroxy, 4 metoxyphenylacetic acid | — | (2.95 ± 0.32) × 10⁻² | (4.12 ± 0.26) × 10⁻² |
| 3,4-dihydroxy-benzoic acid | (1.43 ± 0.04) × 10⁻² | n.d. | (8.72 ± 0.44) × 10⁻² |
| Citric acid | 11.7 ± 1.7 | 8.7 ± 0.2 | 6.9 ± 0.8 |
| Syringic Acid | (3.56 ± 0.05) × 10⁻² | n.d. | (7.74 ± 1.11) × 10⁻² |
| Gallic acid | n.d. | n.d. | 0.19 ± 0.01 |
| Caffeic acid | n.d. | n.d. | n.d. |

^{a}expressed as mg tyrosol equivalent.

To obtain a product suitable for the market, the transformed OVWs can be subjected to centrifugation, filtration and pasteurization processes. In order to evaluate the effects of these treatments, the phenolic composition of the OLIVENOL™ was compared to that of the VW CR43 used for its production. After the treatments, no significant qualitative (FIG. 20) or quantitative (Table 5) differences were observed in the aqueous phase compositions from both samples. In particular, the increase of the OLIVENOL™ dry weight can be attributed to the presence of vegetable glycerin and vegetable gum used as emulsifiers and sweeteners in the final product. As matter of fact, the total phenolic content was almost the same (Table 5). Moreover, the use of centrifugation and filtration resulted in increased amounts of hydroxytyrosol and citric acid in the studied food supplement. These water soluble molecules can be entrapped by adsorption in the solid olive-mill phase, mainly containing polymeric compounds such as lignin, hemicellulose and cellulose. Extracts from this solid phase, in fact, also after an extensive centrifugation, showed low amounts of hydroxytyrosol and citric acid, about 0.54 and 7.49 μg per mg of dry weight, respectively.

The results also show that the industrial process is highly reproducible and the produced OVWs are stable at room temperature. As matter of fact, the OVW CR21, produced about three years before OVW CR43, revealed a phenolic composition similar to that of the OVW CR43 (Table 5).

### Example 17. Conversion of Vegetation Waters into Hydroxytyrosol-Rich Mixtures by Acidic Hydrolysis

In the OVW collected after centrifugation of the liquid-phase mixture that includes olive oil and vegetation water, a high content of oleuropein and other simple phenols with a higher solubility in aqueous phase were found (Table 6). After 6 months, in the OVWs treated and not treated with citric acid, the oleuropein was semi-quantitatively converted in hydroxytyrosol (Table 6): using an authentical standard, in fact, the occurrence of a hydrolytic process of the oleuropein at the acidic pH of OVW has been demonstrated. Moreover, only a slightly higher concentration of tyrosol was observed after the incubation in both treated and untreated OVWs. The studied OVW was obtained from depitted olives so explaining the lower initial tyrosol amount, being this simple phenol present in the seed and in the stone at levels comparable to those revealed in the pulp. In addition, the absence of the tyrosol derivative nuzhenide, which is exclusively present in the seeds, supports the small increase of the tyrosol amount after the proposed incubation.

TABLE 6
Phenolic compounds in the aqueous phase of vegetation water treated and not treated with citric acid
(1.5%) at time 0 and after 6 months. The data are expressed as mean (n = 4) ± standard deviation.

| | not treated OVW | not treated OVW | treated OVW |
|---|---|---|---|
| Time (months) | 0 | 6 | |
| pH | 4.70 | 4.73 | 3.86 |
| Total phenol content^{a} (mg/ml) | 10.50 ± 0.24 | 7.20 ± 0.16 | 10.16 ± 0.6 |
| Dry weight (mg/ml) | 50.0 ± 5.0 | 65.0 ± 5.0 | 65.0 ± 10.0 |

Phenolic compounds content (mg/ml)

| | | | |
|---|---|---|---|
| Oleuropein^{b} | 10.13 ± 0.49 | 1.42 ± 0.72 | 1.88 ± 0.80 |
| Tyrosol | 0.14 ± 0.01 | 0.20 ± 0.03 | 0.18 ± 0.02 |
| Vanillic acid | (5.61 ± 0.39) × 10⁻² | 0.46 ± 0.08 | 0.77 ± 0.04 |
| Hydroxytyrosol | n.d. | 1.32 ± 0.22 | 1.42 ± 0.12 |
| 3-hydroxy, 4-metoxy-phenylacetic acid | 0.20 ± 0.05 | 0.56 ± 0.06 | (2.23 ± 0.41) × 10⁻² |
| 3,4-dihydroxybenzoic acid | 4.73 ± 0.75 | (8.44 ± 0.83) × 10⁻² | 0.44 ± 0.08 |
| Citric acid | — | 1.01 ± 0.05 | 9.3 ± 1.5 |
| Syringic Acid | 3.35 ± 0.72 | 0.38 ± 0.16 | n.d. |
| Gallic acid | (2.77 ± 0.97) × 10⁻² | (4.70 ± 0.37) × 10⁻² | (7.33 ± 0.58) × 10⁻² |
| Gentisic acid | — | (1.76 ± 0.52) × 10−2 | n.d. |

^{a}expressed as mg tyrosol equivalent.
^{b}calculated by HPLC analyses.

After 6 months of incubation in the absence and in the presence of citric acid, a significant decrease in the concentrations of both 3,4-dihydroxybenzoic and syringic acids was observed, whereas the amount of vanillic acid increased, especially in the treated OVW. On the other hand, the content of 3-hydroxy, 4-methoxy-phenylacetic acid increased in the untreated OVW but decreased in the presence of citric acid (Table 6). In the analyzed mixtures, the hydroxytyrosol production has prevalently involved the acid-catalyzed hydrolysis of oleuropein. However, the grown of yeast and fungi in the OVW after the incubation in acidic environment (data not shown), indicate that metabolic processes occur, even if they seem not involved in the hydroxytyrosot formation,

It is known that several aerobic bacterial strains capable of degrading several of the monocyclic aromatic compounds, had been isolated in the OVWs. In particular, specific enzyme analyses have showed the occurrence of o-demethylating activities that can explain the lower content of syringic acid after the proposed incubation. Furthermore, a demonstrated ring-cleavage activity towards 3,4-dihydroxybenzoic acid in strains from OVWs supports the decreased amount of this simple phenol in these experiments. Moreover, the chemical and/or enzymatic degradation of lignin can support the higher content of vanillic acid after the incubation at acidic pH.

On the other hand, the total phenol amount in the sample incubated in the presence of citric acid (Table 6) indicates that the OVWs are stable in acidic environment. The presence of citric acid, as demonstrated for the ascorbic acid, can contribute to keep the phenols concentration constant avoiding the quinones accumulation and the formation of insoluble polymers, due to the direct oxidation by molecular oxygen. In the OVWs, these polymeric phenolic compounds give the sludge characterized by a recalcitrant brownish black color. After the addition of citric acid, an immediate significant decolorization of the OVW persisting during the incubation time, and no increase of solid residue was revealed, indicating an effective degradation of the polymeric fraction. With regard to quinone accumulation in the presence of citric acid, a higher amount of hydroxytyrosol, 3,4-dihydroxybenzoic acid and gallic acid, compounds with chemical structures more susceptible to the oxidation due to the presence of two or more hydroxyl groups were observed (Table 6).

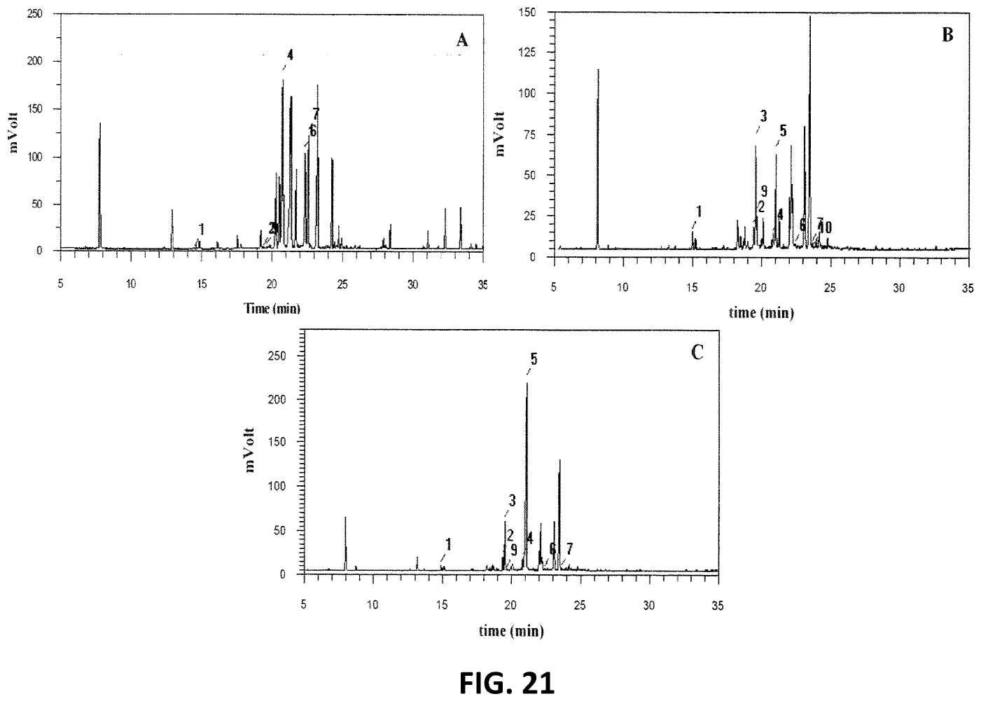

The calculated concentrations of phenolic compounds after 6 months of incubation in acidic environment (Table 6) were similar to those calculated in the OVWs CR43 and CR21 (Table 5), indicating that after this time the OVW transformation is almost over, and supporting the hypothesized stabilizing effect of the citric acid. Even if the phenols composition determined for the treated and untreated OVWs after 6 months of incubation (Table 6) seems quite similar, the advantage in using citric acid to transform the OVW is pointed out by the qualitative GC profiles of both samples (FIG. 21). In the presence of the organic acid, a number of peaks (FIG. 21, panel C) lower than observed in untreated OVW were identified (FIG. 21, panel B): These results, reflected in a higher total phenol content in about the same dry weight (Table 6), suggest that when using citric acid, a mixture can be produced that is enriched in phenols with reduced concentrations of non-phenolic compounds. In order to verify this, GC-MS analyses were carried out to characterize the composition of the treated and the untreated OVWs after the incubation. By spectrum matching and library searching in the NIST/EPS/NIH Mass Spectral Library Database, two small peaks with the m/z 280 fragment related to aglyconic β-phenyl ethanol derivatives of hydroxytyrosol were identified only in the treated OVW, and four significant peaks containing the m/z 280 and 193 fragments, consistent with oleuropein aglyconic forms.

Furthermore, by spectrum matching and library searching, several non-phenolic compounds were identified, especially carbohydrates and secondary metabolites, which is consistent with the occurrence of metabolic processes in treated and untreated OVWs (Table 7).

TABLE 7
Compounds identified in the aqueous phase of vegetation water treated
and not treated with citric acid (1.5%). The hints of the identification
for each compound have a probability higher than 70%.

| | not treated OVW | not treated OVW | treated OVW |
|---|---|---|---|
| Time (months) | 0 | 6 | |
| D-fructose | ++ | − | − |
| Glucofuranoside | ++ | − | − |
| Glucopiranoside | ++ | − | − |
| Dihydroxyacetone | ++ | − | − |
| Malic acid | + | − | ++ |
| Trihydroxy-butirric acid | + | − | ++ |
| Glucitol | + | ++ | ++ |
| Glycerol | − | + | ++ |
| Propanoic acid | − | − | + |
| Xylonic acid | − | ++ | + |
| Xylulose | − | ++ | − |
| Xylitol | − | ++ | − |
| Arabinose | − | ++ | − |
| Ribose | − | ++ | + |
| Deoxyribose | − | ++ | + |
| Galactopyranoside | − | ++ | ++ |
| Ketogluconic acid | − | ++ | − |
| Galactofuranose | − | ++ | − |

− compounds absent
+ compounds present at low concentrations
++ compounds present at high concentrations

In the OVW at time 0, the sugars determined in high concentrations were fructose and glucose aqueous derivatives, whereas the dihydroxyacetone (in both phosphorylated and unphosphorylated forms) was the main secondary metabolite. It is believed that these compounds are metabolized in the untreated OVW in the 5-carbon pentose sugars. These molecules are natural intermediate products, which regularly occur in the glucose metabolism of microorganisms. In particular, compounds such as xylulose, arabinose, ribose and their derivatives such as xylitol and deoxyribose, have been already identified in OVWs and are present in high amount after 6 months of incubation in untreated OVWs (Table 7). As mailer of fact, in the GC-MS spectrum of the untreated OVW peaks attributable to mannose and its derivatives were identified but the probability of the identification was less than 70%. On the other hand, the high concentration of fructose, glucose, and dihydroxyacetone at time 0 can also activate the production of the identified galactose derivatives and glycerol (Table 7) from the glucose metabolism.

A significant difference in the composition of the pentose sugars was observed after the incubation in the presence of citric acid (Table 7), indicating a possible alternative pathway for the glucose degradation. Moreover, higher concentrations of glycerol and trihydroxy-butyric acid revealed after 6 months (Table 7), according with an initial high amount of dihydroxyacetone, suggest the occurrence of a significant lipolysis. These data may indicate the occurrence of different metabolic pathways in the treated and untreated OVWs. At the moment, the presence of a different microorganism flora in the OVWs exposed to the two studied conditions of incubation cannot be excluded.

### Example 18. Inhibitory Effects of Olivenol Plus® Essence Liquid and HIDROX® on Suppression of SARS-CoV-2 Proliferation

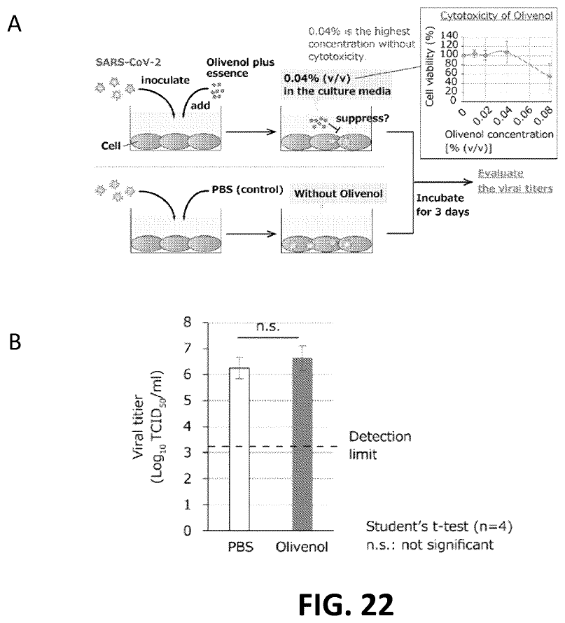

Inhibitory effects of Olivenol Plus® Essence Liquid and HIDROX® on suppression of SARS-CoV-2 proliferation was evaluated. FIG. 22, panel A, shows an experimental protocol for evaluating inhibitory effects of Olivenol Plus® Essence Liquid on suppression of SARS-CoV-2 proliferation in cells. FIG. 22, panel B shows the results of the experiment in FIG. 22, panel A.

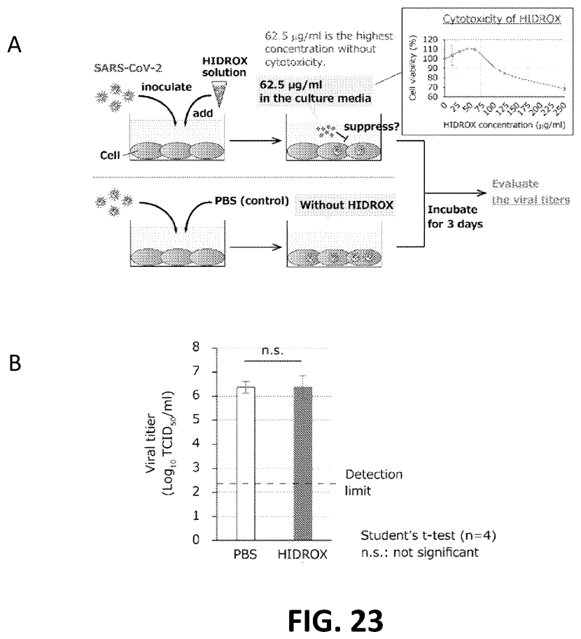

FIG. 23, panel A shows an experimental protocol for evaluating inhibitory effects of HIDROX® on suppression of SARS-CoV-2 proliferation in cells. FIG. 23, panel B shows the results of the experiment in FIG. 23, panel A.

### Example 19. Antiviral Activity of Cells Pre-Treated with Olivenol Plus® Essence Liquid and HIDROX®

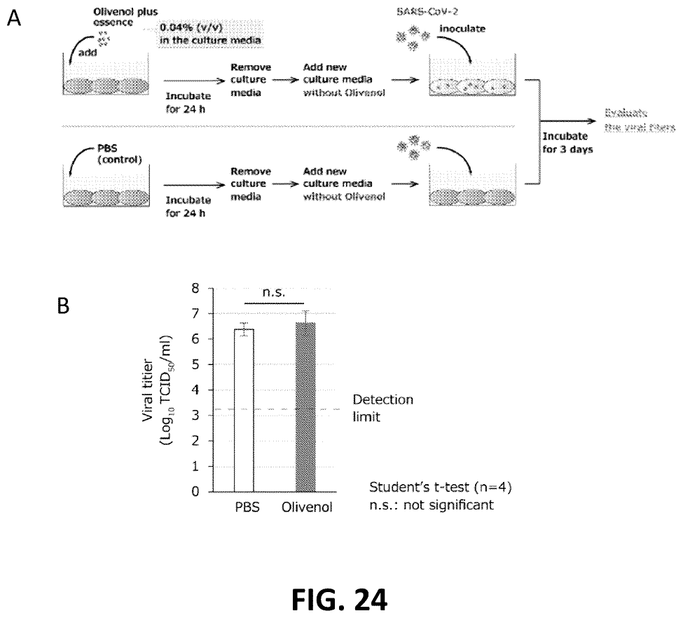

Antiviral activity of cells pre-treated with Olivenol Plus® Essence Liquid and HIDROX® was evaluate. FIG. 24, panel A shows an experimental protocol for evaluating antiviral activity of cells pre-treated with Olivenol Plus® Essence Liquid. Suppression of SARS-CoV-2 proliferation in cells was determined. FIG. 24, panel B shows the results of the experiment in FIG. 24, panel A.

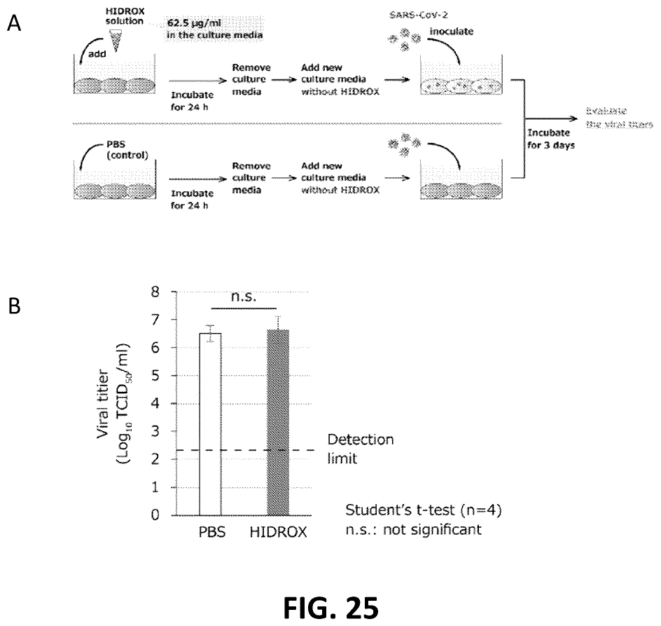

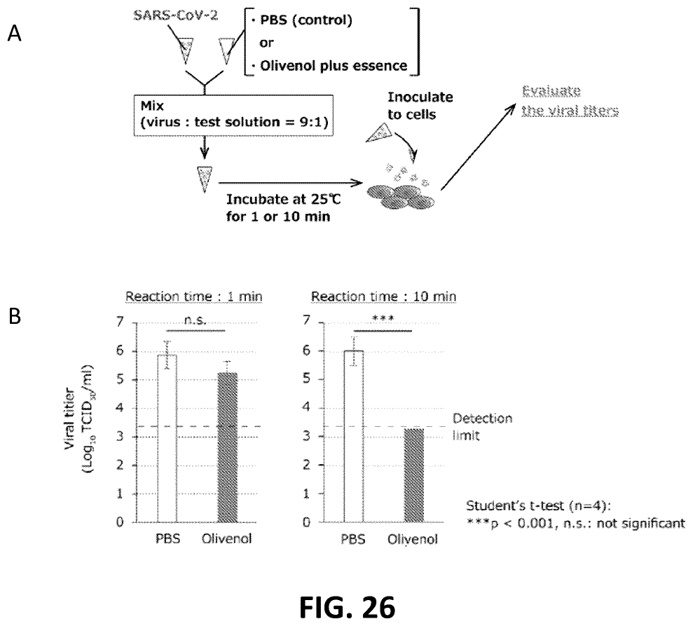

FIG. 25 shows an experimental protocol for evaluating antiviral activity of cells pre-treated with HIDROX®. Suppression of SARS-CoV-2 proliferation in the cells was determined. FIG. 25, panel B shows the results of the experiment in FIG. 26, panel A.

### Example 20. Virucidal Activity of Olivenol Plus® Essence Liquid and HIDROX® on Suppression of SARS-CoV-2 Proliferation

FIG. 26, panel A shows an experimental protocol for evaluating direct virucidal activity of Olivenol Plus® Essence Liquid on suppression of SARS-CoV-2 proliferation in cells. FIG. 26, panel B shows the results of the experiment in FIG. 26, panel A.

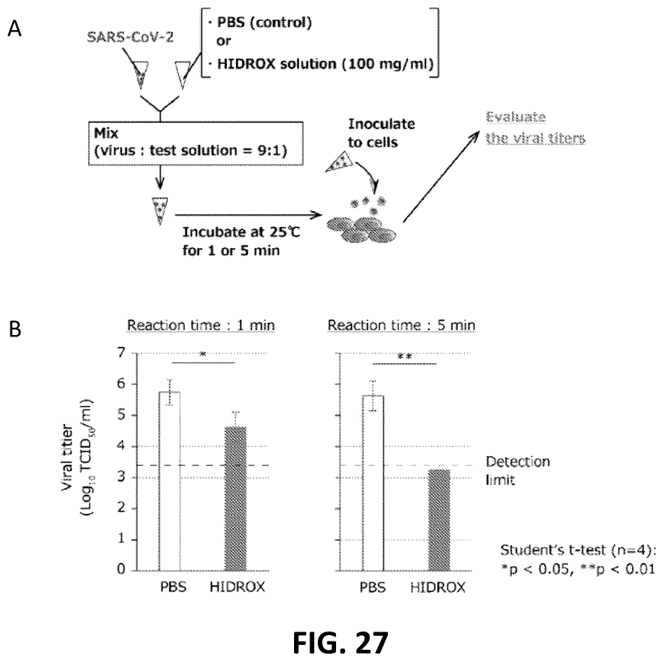

FIG. 27, panel A shows an experimental protocol for evaluating direct virucidal activity of HIDROX® on suppression of SARS-CoV-2 proliferation in cells. FIG. 27, panel B shows the results of the experiment in FIG. 27, panel A.

### Example 21. Effects of Acidic pH on HIDROX®'s Virucidal Activity in Suppressing SARS-CoV-2 Proliferation

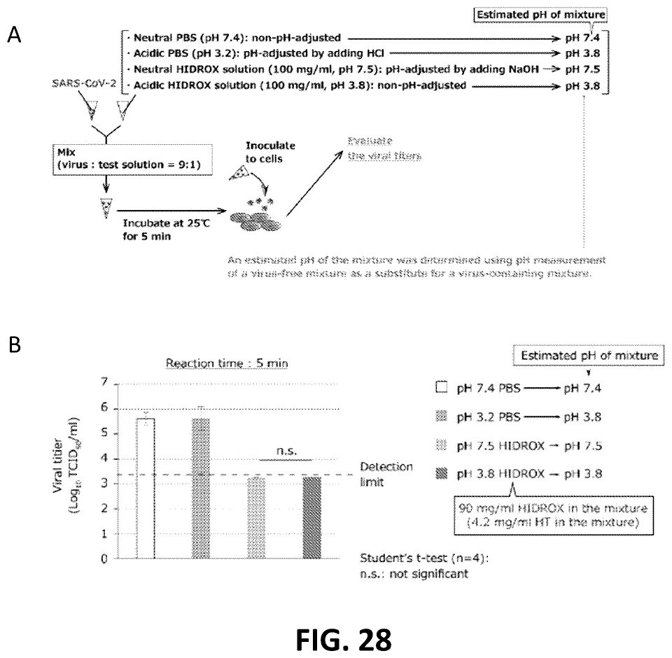

FIG. 28 shows an experimental protocol for evaluating the effects of acidic pH on HIDROX®'s virucidal activity in suppressing SARS-CoV-2 proliferation in cells. FIG. 28, panel B shows the results of the experiment in FIG. 28, panel A.

### Example 22. Effects of HIDROX® in Suppressing Proliferation of the Delta Variant of SARS-CoV-2

Delta variant of SARS-CoV-2 [hCoV-19/Japan/TY11-927-P1/2021] was provided by National Institute of Infectious Diseases (Tokyo, Japan). VeroE6/TMPRSS2 cell, which was established in the National Institute of Infectious Diseases, was obtained from the Japanese Collection of Research Bioresources (Osaka, Japan, Cell No. JCRB1819). For passaging, VeroE6/TMPRSS2 cells were cultured in the growth medium: Dulbecco's Modified Eagle's medium (DMEM; Nissui Pharmaceutical Co., Ltd., Tokyo, Japan) supplemented with 10% fetal bovine serum, 2 mM L-glutamine (Wako Pure Chemical Industries, Ltd., Osaka, Japan), 0.15% NaHCO₃(Wako Pure Chemical Industries, Ltd.), 2 μg/ml amphotericin B (Bristol-Myers Squibb Co., NY, USA), and 100 μg/mL kanamycin (Meiji Seika Pharma Co., Ltd., Tokyo, Japan). After inoculation with SARS-CoV-2, VeroE6/TMPRSS2 cells were cultured in viral growth medium (VGM) composed of DMEM supplemented with 1% fetal bovine serum, 2 mM L-glutamine, 0.15% NaHCO₃, 2 μg/mL amphotericin B, and 100 μg/mL kanamycin. SARS-CoV-2 was handled in the biosafety level 3 facility. The viral titer of the stock solution (VGM) containing SARS-CoV-2) was ˜6.9 log 10 50% tissue culture infective dose (TCID₅₀)/mL.

Test Solutions

The 1.0 g of HIDROX® 12% (Oliphenol LLC., CA, USA) was dissolved in 10

mL of phosphate-buffered saline (PBS), and a centrifugation (850×g, 10 min) was performed. The aqueous layer collected was stored at −30° C. as the stock of 100 mg/mL HIDROX solution. The 1.0 mg/mL of HIDROX solution was prepared by dilution of the stock solutions with PBS. PBS was used as a control solution.

Evaluation of Direct Virucidal Activity of Test Solutions

Viral solution (VGM containing Delta variant of SARS-CoV-2) was mixed with nine volumes of 1.0 mg/mL of HIDROX solution. The final concentrations of HIDROX in the mixture was 0.9 mg/mL. As a diluent control, PBS was mixed with the viral solution. The mixtures were incubated at 25° C. for 3 or 24 h and then inoculated into the cells, and a tenfold serial dilution was performed. After 3 d incubation of the cells at 37° C., a cytopathic effect was observed, and the viral titer (log 10 TCID50/mL) was calculated using the Behrens-Kärber method. The detection limit of the viral titer in each test solution group was determined based on the cytotoxicity of each test solution. The detection limit of the viral titer in the group treated by PBS and HIDROX solution was set to 1.25 and 2.25 log 10 TCID50/mL, respectively.

Statistical Analysis

Student's t test was performed to determine statistically significant differences between PBS and HIDROX groups. Statistical significance was set at p<0.05.

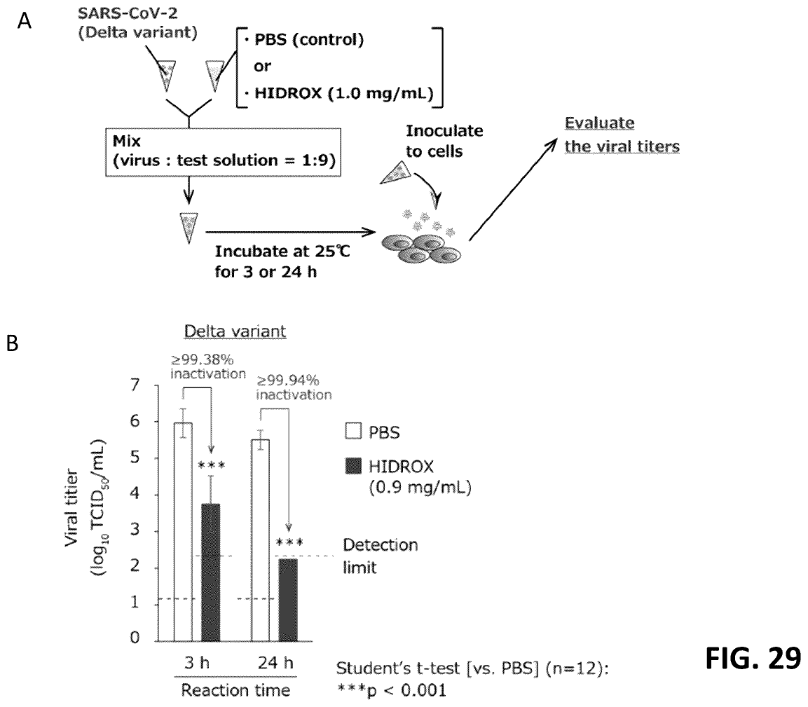

99| FIG. 29, panel A shows the experimental protocol for evaluating the effects of HIDROX on the proliferation of the Delta variant of SARS-CoV-2 in cells. FIG. 29, panel B shows the results of the experiment in FIG. 29, panel A.

1. A method for preventing, treating or ameliorating a symptom of a viral infection in a subject, comprising the step of:
administering to the subject an effective amount of an olive component composition, wherein the olive component composition comprises one or more polyphenyl compounds selected from the group consisting of hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof.

2. The method of claim 1, wherein the olive component composition comprises hydroxytyrosol and one or more other polyphenyl compounds.

3. The method of claim 1, wherein the olive component composition further comprises one or more non-phenolic compounds selected from the group consisting of D-fructose, glucofuranoside, glucopyranoside, dihydroxyacetone, malic acid, trihydroxybutyric acid, glucitol, glycerol, propanoic acid, xylonic acid, xylulose, xylitol, arabinose, ribose, deoxyribose, galactopyranoside, ketogluconic acid and galactofuranose.

4. The method of claim 1, wherein the olive component composition is formulated for oral administration.

5. The method of claim 1, wherein the olive component composition is formulated for topical administration.

6. The method of claim 1, wherein the olive component composition is formulated for inhalation.

7. The method of claim 1, wherein the olive component composition comprises acid-treated olive juice.

8. The method of claim 1, wherein the subject is infected with or at the risk of being infected with SARS-CoV-2 virus.

9. The method of claim 1, wherein the subject is infected with or at the risk of being infected with influenza virus.

10. A method for preventing or reducing surface transmission of a virus, comprising the step of:
applying an effective amount of an olive component composition to a surface for a period of time to inactivate a virus on the surface,
wherein the olive component composition comprises one or more polyphenyl compounds selected from the group consisting of hydroxytyrosol, tyrosol, tyrosol esters of elenolic acid, such as oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof.

11. The method of claim 10, wherein the olive component composition comprises acid-treated olive juice.

12. The method of claim 10, wherein the virus is SARS-CoV-2 virus or influenza virus.

13. An antiviral composition, comprising:
an olive component composition, wherein the olive component composition comprises:
(1) one or more polyphenyl compounds selected from the group consisting of hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof; and
(2) propanoic acid.

14. The antiviral composition of claim 13, wherein the olive component composition does not comprise syringic acid, or comprises syringic acid in an amount of less than 0.01 mg/ml.

15. An antiviral composition, comprising an olive component composition, wherein the olive component composition comprises: one or more polyphenyl compounds selected from the group consisting of hydroxytyrosol, tyrosol, tyrosol esters of elenolic acid, oleuropein, demethyloleuropein, oleuropein aglycone (3,4 DHPEA-EA), oleocanthol (HPEA-EDA), ligstroside, lingstroside aglycone (3,4 DHPEA-EA), 10-hydroxyoleuropein, and 10-hydroxyligstroside; vanillic acid, 3-hydroxy, 4-methoxyphenylacetic acid, 3,4-dihydroxy-benzoic acid, citric acid, syringic acid, gallic acid, caffeic acid, gentisic acid, 3,4 DHPEA-EDA, lignanes, flavonoids, elenolic acid, alpha-tocopherol, verbascoside, pinoresinol lignan, rutin flavonoid, secoiridoids, pinoresinol, and lycosylated, deglycosylated, phosphorylated and dephosphorylated forms thereof,
wherein the antiviral composition is formulated in an aerosol for inhalation.
